(19) United States
(12) Patent Application Publication
(10) Pub. No.: US 2025/0065126 A1
(43) Pub. Date: Feb. 27, 2025

(54) SYSTEMS, DEVICES, AND METHODS FOR PERTUBING A BIOSYSTEM TO QUANTIFY RESPONSIVENESS

(71) Applicants: John Cornwell, San Francisco, CA (US); SRI International, Menlo Park, CA (US)

(72) Inventors: Christopher Ian Connolly, Meno Park, CA (US); Patrick Denis Lincoln, Menlo Park, CA (US); John Cornwell, San Francisco, CA (US)

(73) Assignee: SRI International, Menlo Park, CA (US)

(21) Appl. No.: 18/810,905

(22) Filed: Aug. 21, 2024

Related U.S. Application Data
(60) Provisional application No. 63/534,058, filed on Aug. 22, 2023.

(51) Int. Cl. A61N 1/36

(52) U.S. Cl. CPC A61N 1/36135; A61N 1/36053

(57) ABSTRACT

Embodiments are directed to systems, devices, and methods for perturbing a biosystem to quantify responsiveness. An example system comprises stimulation circuitry configured to output a plurality of biostimulation signals to a target of a subject, sensor circuitry configured to obtain measures of a biosignal from the subject, and processor circuitry. The processor circuitry is configured to cause the stimulation circuitry to output the plurality of biostimulation signals to perturb a biosystem of the subject, and quantify responsiveness of the biosystem to the perturbation based on the plurality of biostimulation signals and measures of the biosignal responsive to the plurality of biostimulation signals applied to the target.

### BACKGROUND

Nerve signals may be stimulated (induced, modified, and/or interrupted) using stimulation circuitry. For example, measured peripheral nerve tissue signals can be processed into synthetic neuromodulation signals which can be generated and applied to tissue of a subject for various applications, including but not limited to, therapeutic treatment. A non-limiting example nerve is the vagus nerve, which is located on each side of the human body. The vagus nerve is a component of the autonomic nervous system and plays roles in metabolic and physiologic homeostasis.

Different types of stimulation devices have been used to stimulate nerves or other tissue. Examples include implantable devices that stimulate different tissue for treatment of varying conditions, including heart disease, epilepsy, and depression. These devices are typically implanted through surgery by subcutaneously placing a generator in the upper chest of a patient. An electrode lead is then attached from the generator to the tissue. Other types of devices include transcutaneous stimulation devices. For example, transcutaneous stimulation devices can be used to stimulate the auricular branch of the vagus nerve by targeting the cutaneous receptive field of the auricular branch of the vagus nerve.

### SUMMARY

The present invention is directed to systems, devices, and methods for perturbing a biosystem to quantify responsiveness.

Various embodiments of the present disclosure are directed to a system comprising stimulation circuitry configured to output a plurality of biostimulation signals to a target of a subject, sensor circuitry configured to obtain measures of a biosignal from the subject, and processor circuitry configured to: cause the stimulation circuitry to output the plurality of biostimulation signals to perturb a biosystem of the subject; and quantify responsiveness of the biosystem to the perturbation based on the plurality of biostimulation signals and measures of the biosignal responsive to the plurality of biostimulation signals applied to the target.

In some embodiments, the system further includes memory circuitry in communication with the processor circuitry which stores a depository of the plurality of biostimulation signals, wherein each of the plurality of biostimulation signals represent a processed tissue signal as a sequence of at least one state corresponding to a set of state parameters and correlated with causing a particular physiological effect of perturbing the biosystem.

In some embodiments, the processor circuitry is configured to: project the measures of the biosignal received from the sensor circuitry to provide a trajectory along a shape in phase space representing a biosignal cycle of the biosignal; and based on clusters in the trajectory along the shape, quantify the responsiveness of the biosystem to the perturbation.

In some embodiments, the processor circuitry is configured to project the measures of the biosignal received from the sensor circuitry onto a Fourier basis to provide the trajectory along a torus in phase space representing the biosignal cycle of the biosignal.

In some embodiments, the processor circuitry is configured to output an indication of homeostatic context based on a size and angle of the clusters.

In some embodiments, the processor circuitry is configured to quantify the responsiveness of the biosystem for the subject over time and based on feedback, wherein the feedback is selected from at least one of: additional measures of the biosignal, other physiological data, behavioral activity of the subject, environmental activity of the subject, and a combination thereof.

In some embodiments, the processor circuitry is configured to strobe the measures of the biosignal at a particular frequency.

In some embodiments, the plurality of biostimulation signals include different values for a stimulation parameter, wherein the stimulation parameter is selected from at least one of: pulse frequency, duration, amplitude, duty cycle, pulse width, delivery portal, and a combination thereof.

In some embodiments, the processor circuitry includes a machine learning model which is trained using the plurality of biostimulation signals and measures of the biosignal to identify a transfer pattern of stimulation parameters that optimize an effect associated with the biosignal, and predict the biosignal response and a homeostatic state, wherein the system outputs an indication of the biosignal response and the homeostatic state.

In some embodiments, the plurality of biostimulation signals include a plurality of neuromodulation signals applied to a nerve target of the subject.

In some embodiments, the processor circuitry is configured to establish a stimulus program that causes the stimulation circuitry to output an additional plurality of biostimulation signals to the subject or other subjects and timing for the additional plurality of biostimulation signals to achieve a goal associated with a homeostatic state.

In some embodiments, the processor circuitry includes a machine learning model which is trained to: identify a first transfer pattern that maps the measures of a second biosignal and the plurality of biostimulation signals, identify a second transfer pattern that maps the measures of the biosignal and the second biosignal; and input the biosignal, as a proxy for the second biosignal, to the machine learning model and to predict an effect of an additional biostimulation signal on the second biosignal.

Various embodiments of the present disclosure are directed to a method comprising applying a plurality of biostimulation signals to a target of a subject to perturb a biosystem of the subject, receiving measures of a biosignal from the subject responsive to the plurality of biostimulation signals applied to the target, and quantifying responsiveness of the biosystem to the perturbation based on the plurality of biostimulation signals and the measures of the biosignal.

In some embodiments, quantifying the responsiveness of the biosystem includes providing a trajectory of the measures of the biosignal along a shape in phase space representing a biosignal cycle of the biosignal and quantifying the responsiveness of the biosystem to the perturbation based on clusters in the trajectory along the shape.

In some embodiments, quantifying the responsiveness of the biosystem includes: providing the trajectory along a torus in phase space representing the biosignal cycle of the biosignal by projecting the measures of the biosignal onto a Fourier basis; and quantifying the responsiveness of the biosystem to the perturbation based on clusters in the trajectory along the torus.

In some embodiments, the method includes identifying a transfer pattern of stimulation parameters that optimize an effect associated with the biosignal using a machine learning model which is trained using the plurality of biostimulation signals and the measures of the biosignal.

In some embodiments, the method includes downloading the plurality of biostimulation signals from external memory circuitry, wherein each of the plurality of biostimulation signals represent a processed tissue signal as a sequence of at least one state corresponding to a set of state parameters and correlated with causing a particular physiological effect of perturbing the biosystem.

In some embodiments, the method includes receiving baseline measures of the biosignal without biostimulation applied and comparing the baselines measures to the measures of the biosignal responsive to the plurality of biostimulation signals applied to the target to quantify the responsiveness of the biosystem.

In some embodiments, the method includes receiving measures of a second biosignal from the subject and using a machine learning model to: identify a first transfer pattern that maps the measures of the second biosignal and the plurality of biostimulation signals; and identify a second transfer pattern that maps the measures of the biosignal and the second biosignal. The method further includes inputting the biosignal, as a proxy for the second biosignal, to the machine learning model and to predict an effect of an additional biostimulation signal on the second biosignal.

Various embodiments are directed to a non-transitory computer-readable storage medium comprising instructions that when executed cause processor circuitry to: cause stimulation circuitry to output a plurality of biostimulation signals to a target of a subject to perturb a biosystem of the subject; receive measures of a biosignal from the subject responsive to the plurality of biostimulation signals; and quantify responsiveness of the biosystem to the perturbation based on the plurality of biostimulation signals and the measures of the biosignal responsive to the plurality of biostimulation signals.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

Various example embodiments can be more completely understood in consideration of the following detailed description in connection with the accompanying drawings, in which:

FIG. 1 illustrates an example system for perturbing a biosystem to quantify responsiveness, in accordance with the present disclosure.

FIGS. 2A-2B illustrate example biostimulation signals and measures of a biosignal responsive thereto, in accordance with the present disclosure.

FIG. 3 illustrates an example method for quantifying responsiveness of a biosystem of a subject, in accordance with the present disclosure.

FIG. 4 illustrates an example computing device including non-transitory computer-readable storage medium storing executable instructions, in accordance with the present disclosure.

FIG. 5 illustrates an example machine learning process, in accordance with the present disclosure.

FIG. 6 illustrates an example system including machine learning models, in accordance with the present disclosure.

FIGS. 7A-7D illustrate example machine learning models, in accordance with the present disclosure.

FIG. 8 illustrates an example of a plurality of biostimulation signals used to perturb a biosystem of a subject, in accordance with the present disclosure.

FIGS. 9A-9C illustrate example biosignal measures responsive to a plurality of biostimulation signals, in accordance with the present disclosure.

FIG. 10 illustrates example clusters of biosignal measures as projected in a trajectory along a torus, in accordance with the present disclosure.

FIG. 11 illustrates an example of unwrapping the torus of biosignal measures, in accordance with the present disclosure.

FIG. 12 illustrates an example of advancing the strobe of the biosignal measures, in accordance with the present disclosure.

FIG. 13 illustrates example biosignal measures when stimulating on different sides of a subject, in accordance with the present disclosure.

### DETAILED DESCRIPTION

In the following detailed description, reference is made to the accompanying drawings which form a part hereof, and in which is shown by way of illustration specific examples in which the disclosure can be practiced. It is to be understood that other examples can be utilized, and various changes may be made without departing from the scope of the disclosure. The following detailed description, therefore, is not to be taken in a limiting sense, and the scope of the disclosure is defined by the appended claims. It is to be understood that features of the various examples described herein may be combined, in part or whole, with each other, unless specifically noted otherwise.

Embodiments in accordance with the present disclosure involve a system, device, and/or method for using the peripheral nervous system or other biosystem for presenting and probing the homeostatic state of the biosystem of a subject, such as the cardiovascular system. Such biosystems can be thought of as a coupled oscillator (e.g., heart, lungs, stomach, intestine, hypothalamus) that operate at a certain rate. Measurements of the biosystems, e.g., biosignal measures, can be performed directly or indirectly, such as by using heart monitors, implantable sensor devices, and/or wearable devices. In some embodiments, the biosystems can be probed using the peripheral nervous system by stimulating a nerve (e.g., the auricular vagus nerve). Example systems, devices, and/or methods can use constructed probes in the form of stimulus programs and schedules, and measures the responses of the biosystem to assess health status by projecting the biosignal measures to a shape (e.g., torus) on an appropriate phase space to represent the behavior of the biosystem. In some embodiments, a depository of a plurality of biostimulation signals can be used, which represent processed signals as a sequence of at least one state corresponding to a set of state parameters and causing a particular effect. In some embodiments, the plurality of biostimulation signals can be implemented to include at least some of substantially the same features and attributes as described by U.S. Pat. No. 11,752,339, issued on Sep. 12, 2023, and entitled “Methods and Systems for Stimulating Nerve Signals”, which is incorporated herein in its entirety for its teaching. For example, applying an electrical signal, as compared to an acoustic signal, to the left and/or right cymba conchae that is above the sensory threshold, but below the pain threshold, can result in brain activation that is similar to that of the left and/or right cervical vagus nerve stimulation.

Prior approaches for assessing the homeostatic state of a biosystem are open-loop, i.e., measure the biosystem of concern, collect baseline data, and attempt to identify anomalous conditions. An open-loop technique misses a large attack surface for pathology. By analogy to cybersecurity, embodiments of the present disclosure use a plurality of biostimulation signals to perturb (e.g., fuzz) the biosystem and observe the response of the biosystem to perturbations that may be considered to be medically benign, such as but not limited to transcutaneous auricular vagus nerve stimulation (taVNS). In particular, example systems, devices, and methods measure for baseline responses, perturbs the biosystem, and then observes the perturbed response. From this, the biosystem responsiveness and resistance to perturbation can be quantified. In some embodiments, different perturbations can yield different responses, providing a wealth of data.

In various embodiments, biosignal measures are collected and projected onto a Fourier basis set that describes responses in terms of trajectories along a torus or other shape in phase space. These trajectories can cluster in response to biostimulation and can reveal information about how the biosystem responds to perturbations. For example, taVNS can modify heart rate (HR) and heart rate variability (HRV). HR can be slowed down or sped up depending on the side of stimulation (e.g., left verses right). The degree of increase in HR or HRV can be controlled by adjusting the pulse width of the biostimulation signal, among other stimulation parameters.

In some embodiments, the biosignal measures can be strobed with respect to a particular frequency of a feature (e.g., a peak or trough in the biosignal), as further described herein. For example, for observing the cardiac biosystem, the biosignal measures can be strobed at approximately 0.9 Hertz (Hz) due to an unobserved lag in cardiac cycle trajectories. By strobing the data, the effect of biostimulation (e.g., VNS) can be visualized by cluster points and by stimulation parameters.

In some embodiments, the systems, devices, and/or methods can have an immediate impact in biosystem health, such as in the cardiovascular health space. Current approaches focus on anomalies in the cardiac cycle and represent imminent health concerns. In contrast, systems, devices, and/or methods of the present disclosure are akin to taking blood pressure measurements. By sounding cardiovascular health, non-invasive finer control and longer-term detection can be achieved, as well as prediction of cardiovascular pathologies. Embodiments are not limited to the cardiac biosystem and can be applied to other non-cardiac biosystems as well as other forms of external stimulation.

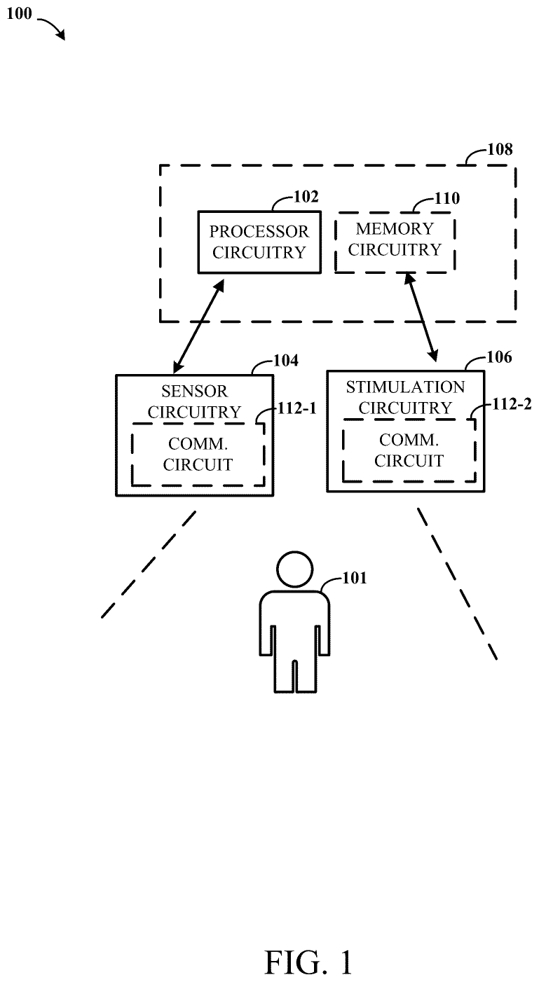

Turning now to the figures, FIG. 1 illustrates an example system for perturbing a biosystem to quantity responsiveness, in accordance with the present disclosure. The system 100 includes processor circuitry 102, sensor circuitry 104, and stimulation circuitry 106.

The stimulation circuitry 106 can output a plurality of biostimulation signals to a target of a subject 101. The stimulation circuitry 106 can include a generator configured to generate the biostimulation signals, such as synthetic neuromodulation signals, which emulates stimulation on a target. A biostimulation signal includes and/or refers to a signal output to stimulate the target of the subject. The biostimulation signals can be waveforms that are delivered with particular stimulation parameters. For example, the generator can deliver the biostimulation to the target at a particular rate and power. The stimulation circuitry 106 can use known technologies to apply the biostimulation signals to the subject 101, including electrical, electromechanical, optical, and acoustic technologies, among others. For example, the stimulation circuitry 106 can include various types of generators, such as but not limited to electrodes, light emitting diodes or other light-emitting devices, mechanical vibrators, radio-frequency transducers, electromagnets, and/or other mechanical or electromechanical components, which can be implemented on various devices, such as speakers, headphones, ear buds, chest straps, smart eye coverings (e.g., glasses, goggles), virtual reality headsets, among others.

As shown, the stimulation circuitry 106 can include communication circuitry 112-2, which provides for communication between the stimulation circuitry 106 and the processor circuitry 102. While not illustrated, the processor circuitry 102 or a computing device 108 that includes the processor circuitry 102 can also include communication circuitry. As further described below, the processor circuitry 102 can communicate with the stimulation circuitry 106 to cause output of biostimulation signals. In some embodiments, the stimulation circuitry 106 can form part of a computing device 108 with the processor circuitry 102, and in other embodiments, the stimulation circuitry 106 forms part of a device that is separate from the computing device 108 and/or the processor circuitry 102. For example, the stimulation circuitry 106 can form part of a wearable device, such as a headset, headphones, or ear buds, which can be worn in stimulating proximity to the target of the subject 101. In such embodiments, the processor circuitry 102 can form part of the computing device 108, such as a smartphone, tablet, or laptop computer that is in communication with the wearable device.

In some embodiments, the plurality of biostimulation signals can be stored on a depository, which can be stored on local memory of or associated with the processor circuitry 102 (e.g., memory circuitry 110) or memory circuitry external to, and in communication with, the processor circuitry 102. For example, the system 100 can further include memory circuitry 110 in communication with the processor circuitry 102 which stores the depository of the plurality of biostimulation signals. The depository includes a storage location for plurality of biostimulation signals and can be local to processor circuitry 102 and/or the computing device 108. As further described herein, each of the plurality of biostimulation signals stored on the depository can represent a processed tissue signal as a sequence of at least one state corresponding to a set of state parameters and correlated with causing a particular physiological effect of perturbing the biosystem.

In various embodiments, the stimulation circuitry 106 and processor circuitry 102 and/or computing device 108 can be implemented as and/or include at least some of substantially the same features and attributes as the neuromodulation signal generator system and electronic device, including the library storing a set of state parameters for generating synthetic neuromodulation signals, as described by U.S. Pat. No. 11,752,339. In some embodiments, the above-described depository can be formed from example libraries as described by U.S. Pat. No. 11,752,339. The library can include a set a biostimulation signals stored in memory circuitry, such as external memory circuitry, that is accessible via a network to form the local depository. A particular subset of the set of biostimulation signals for use in a particular application, e.g., perturbing a biosystem, can be obtained (e.g., downloaded) by the processor circuitry 102. For example, the plurality of biostimulation signals can be downloaded from the library to form a local depository as stored on the memory circuitry 110 and which are associated with the specific effect of perturbing the biosystem of the subject 101 (and/or other effects). Other effects can be identified and, in response, additional biostimulation signals can be downloaded and stored in the depository and applied to the subject 101. The library can be accessed through any suitable network or communications link, including wireless, optical or wired computing systems. Each of the biostimulation signals on the library can describe at least one state along with control data that contains information about how the biostimulation signals can be used. In some embodiments, the processor circuitry 102 can access digital representations of the biostimulation signals from the library and output the digital representations of the biostimulation signals to the stimulation circuitry 106 and the stimulation circuitry 106 can convert the digital representations to the analog domain for application by the generator.

In some embodiments, the plurality of biostimulation signals can include electrical stimulation signals, acoustic stimulation signals, ultrasound stimulation signals, optical stimulation signals, magnetic stimulation signals, or other types of stimulation and various combinations thereof. The stimulation circuitry 106 can generate and output the plurality of biostimulation signals to a target of the subject 101. In some such embodiments, the biostimulation signals can include (synthetic) neuromodulation signals applied to a nerve target. In some embodiments, the nerve target is a peripheral nerve, such as the vagus nerve. However, embodiments are not so limited and other tissue can be targeted. For example, the target can include a nerve, differentiated tissue, cells, a muscle, and/or an organ, among other targets. As some examples, the biostimulation can include stimulating muscles, such as via stimulating efferent nerve fibers (e.g., Transcutaneous electrical nerve stimulation (TENS)) or direct muscles stimulation (e.g., electrical muscle stimulators (EMS)). As another example, ultrasound signals can be applied to internal organs, such as to the spleen or liver. As a further example, transcranial magnetic stimulation can be applied to the brain and/or brainstem. Other biostimulation signals and/or targets can be used.

In some embodiments, the target can be associated with or include a delivery portal (e.g., locations). For example, a portion of the car or cars can be the delivery portal. As other examples, a part of the head, eye(s), and/or chest can be the delivery portal.

In some embodiments, each of the plurality of biostimulation signals can represent at least one processed measured tissue signal (e.g., nerve tissue signal) as a sequence of at least one state represented by at least one state parameter. The state parameters can define the waveform of the measured tissue signal, such as including waveform parameters, amplitude mean and variance, firing rate mean and variance. As further described herein, state parameters defining the measured tissue signal waveform can be adjusted to account for the biostimulation signal transforming when penetrating through skin and other tissue to the target and/or can define or include the stimulation parameters. Said differently, the state can describe a stimulation signal or a recorded tissue signal (e.g., a biosignal). For stimulation, the sequence of states is for a desired effect, with state parameters assigned to each state. For a recorded tissue signal, the signal is captured and translated to the sequence of states represented by the state parameters that fit the recording.

Stimulation of the tissue can be based on biostimulation signals generated based on stimulation parameters. Stimulation parameters include and/or refer to parameters that define the waveform of the biostimulation signals, such as frequency, duty cycle, pulse width, among others. In some embodiments, stimulation of the tissue can be based on newly defined biostimulation signals that are determined to have a beneficial effect on the subject 101. The systems, methods, and devices disclosed herein can enable generation of these stimulus patterns without requiring surgery, or prior recordings of tissue functions. The biostimulation signals can be presented to an individual through a variety of means. For example, the biostimulation signals can be presented through sound vibrations, light stimulation, and other devices attached to the car or eye that are configured to stimulate nerves, such as the vagus nerve.

Embodiments disclosed herein can provide a convenient, safe, and effective way for the development and use of biostimulation techniques. In some embodiments, the technologies described herein can provide personalized health and/or behavioral benefits. In some embodiments, subjects can directly manage their individual health condition through an automated system, which can include a user-friendly human-computer interface, instructions implemented in software, and hardware including processor(s), memory, and input/output device(s). As previously described, some embodiments include a library and/or depository of biostimulation signals, such as synthetic neuromodulation signals. Each biostimulation signal can correspond to a specific pattern that has been correlated with a particular desired effect. For example, stored synthetic biostimulation signals for generating a biostimulation signal (BSS) #1 can be useful to perturb the cardiac system. A subject or other person (e.g., a user) can download the synthetic biostimulation signal from the library, and load it into a computing device 108 or directly onto stimulation circuitry 106 (on the electronic device or stand-alone) configured to stimulate tissue. By playing the BSS #1 on his or her device, the subject 101 can perturb the cardiac system without causing a (medical) condition and/or in a manner that is considered to be medically benign. The depository can include biostimulation signals for a variety of effects (such as perturbing the biosystem), as further described below. The effects can be associated with stimulations of the vagus nerve, or other nerves and/or tissue in the body.

In some embodiments, at a least a portion of the biostimulation signals can include neuromodulation signals. The neuromodulation signals can be generated by measuring a peripheral nerve tissue signal taken from a subject subjected to a condition; creating a synthetic neuromodulation signal by representing at least one of the measured peripheral nerve tissue signals (e.g., neurograms) as a sequence of at least one state, wherein each state is represented by at least one state parameter that is/are converted to the synthetic neuromodulation signal; and sending the synthetic neuromodulation signal to the stimulation circuitry 106 configured to apply the synthetic neuromodulation signal to the subject 101, wherein application of the synthetic neuromodulation signal to the subject 101 causes the subject 101 to experience an intended effect and which may be without application of the condition to the subject 101.

Embodiments are not limited to neuromodulation signals, and the above can be applied to other types of biostimulation. For example, a tissue signal (e.g., brain signal, acoustic signal, EMG or other type of signal) can be recorded from a subject 101 subjected to a condition; following, a synthetic biostimulation signal can be created by representing at least one of the measured tissue signals as a sequence of at least one state, with each state represented by state parameter(s). As a specific example, for acoustic biostimulation, the state parameters can be similar to those for electrical stimulation. For stimulating organs, ultrasound can be used to modulate the behavior of non-neural tissue to achieve the effects in non-neural biosignals (e.g., cytokines). In some such embodiments, a mode of biostimulation can be defined that specifies the kind of energy to be used for biostimulation (e.g., electrical, acoustic, ultrasound) and the target where the energy is to be applied (e.g., cymba, concha, spleen). In some embodiments, the biostimulation can include multi-modal stimulation that is applied in parallel, where the states can specify the mode (e.g., ultrasound verses electrical). In such embodiments, the state machine can diverge (e.g., fork) and transition to two or more states in parallel, one for each mode. The state path(s) can diverge and then join, with divergence and joining indicating when states are executed in parallel.

In various embodiments, the sensor circuitry 104 can obtain measures of a biosignal from the subject 101. Example biosignals include cardiac signals (e.g., HR, HRV, electrocardiogramady or skin temperature data, respiratory data (e.g., respiratory rate, tidal volume), oxygen levels or saturation (e.g., photoplethysmograph (PPG)), blood pressure, brain, muscle, and/or or nerve data (e.g., electromyogram (EMG), electroencephalogram (EEG), electroneurogram (ENG), neural responses which are captured using magnetic resonance imaging (MRI)), skin conductance or galvanic response, hormone levels, among others measures as well as combinations thereof. A biosignal includes and/or refers to a signal measured from a living thing. In some embodiments, the sensor circuitry 104 can collect additional signals. For example, the sensor circuitry 104 can collect biosignals that are continuous and discrete and which are derived from a biological state (e.g., intrinsic) and/or signals derived from an environmental state (e.g., extrinsic). Example signals derived from an environmental state include acoustic signals, temperature, movement signals (e.g., motion, steps), and optic signals, among other signals.

In some embodiments, the sensor circuitry 104 can include wearable technology, such as wearable devices, sensors and/or environmental sensors, among others. Wearable technology, as used herein, includes and/or refers to sensor(s) and/or device with at least one sensor that is wearable and used to obtain a biosignal (e.g., physiological) measurements from the subject 101 or extrinsic measurements derived from the environment. In some embodiments, the wearable technology can be continuously worn by the subject 101 for a period of time or periods of time (e.g., for a day, for multiple days, for months, all day, all night). Non-limiting examples of wearable technology include a smart watch, fitness watch, smart ring, a chest strap, a headset, a headphone or ear bud(s), among others. In some embodiments, alternatively or additionally, the sensor circuitry 104 can include non-wearable sensors and/or other technology, which can be embedded in a particular location and/or environment. Similar to the stimulation circuitry 106, the sensor circuitry 104 can include communication circuitry 112-1, which provides for communication between the sensor circuitry 104 and the processor circuitry 102.

In some embodiments, as noted above, the processor circuitry 102 can form part of a computing device 108. The computing device 108 can further include the memory circuitry 110 that stores instructions executable by the processor circuitry 102. In other embodiments, the processor circuitry 102 and/or the memory circuitry 110 can form a part of distributed computing devices, with the distributed computing devices being in communication with each other.

The processor circuitry 102 can cause the stimulation circuitry 106 to output the plurality of biostimulation signals to perturb the biosystem of the subject 10, and in response, quantify responsiveness of the biosystem to the perturbation based on the plurality of biostimulation signals and measures of the biosignal, e.g., which are responsive to the plurality of biostimulation signals applied to the target. As further described herein, baseline measures of the biosignal can be obtained, which are measured with no biostimulation applied, and are compared to the measures of the biosignal responsive to the perturbation of the biosystem to quantify the responsiveness of the biosystem.

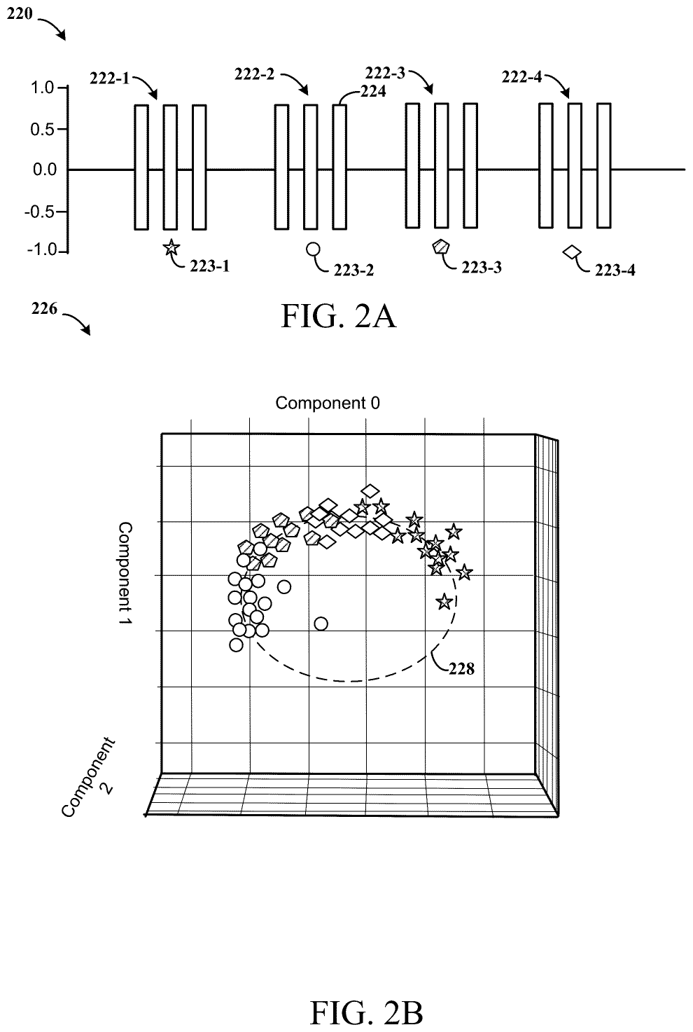

In some embodiments, the plurality of biostimulation signals output to the target can include different values for a stimulation parameter. Example stimulation parameters include pulse frequency, duration, amplitude, duty cycle, pulse width, delivery portal, and a combination thereof. By outputting biostimulation signals with a stimulation parameter at different values to a target of the subject 101, the biosystem can be perturbed and responses to the perturbation can be observed. Example biostimulation signals with the stimulation parameter at different values are further illustrated herein at least by FIG. 2A.

Responsiveness of the biosystem to the perturbation can be indicative of a homeostasis health and/or homeostatic state of the subject 101. Homeostasis includes and/or refers to the body's ability to maintain stability, even with faced with external changes. In homeostasis, the body levels of various biosignals (e.g., acid, blood pressure, blood sugar, electrolytes, energy, hormones, oxygen, respiratory signals, cardiac signals, temperature, proteins) are constantly adjusted to respond to changes inside and outside the body and to keep them at a normal level.

In some embodiments, quantifying responsiveness of the biosystem can include at least one of: (i) changes (e.g., trends or specific values, such as increase or decreasing) in the biosignal responsive to different values of the stimulation parameter; (ii) a specific value of the stimulation parameter which causes the change or a greatest change; and (iii) changes in responsiveness of the biosystem to the perturbation over time. Example changes of the responsiveness of the biosystem can include changes to phase angle and/or cluster variances, as further described herein. Changes in the biosignal response can include different amplitudes or variances. In some embodiments, the quantified responsiveness of the biosystem can include an indication that the biosystem is resistant to the perturbation (e.g., did not respond or no change).

In some embodiments, the processor circuitry 102 can further process the biosignal measures in order to quantify the responsiveness of the biosystem to the perturbation. For example, the processor circuitry 102 can: (i) project the measures of the biosignal received from the sensor circuitry 104 to provide a trajectory along a shape in phase space representing a biosignal cycle of the biosignal, and (ii) based on the clusters in the trajectory along the shape, quantify the responsiveness of the biosystem to the perturbation. In some embodiments, the shape can include a torus and/or the measures can be projected onto a Fourier basis. In some such embodiments, the processor circuitry 102 can project the measures of the biosignal as received from the sensor circuitry 104 onto a Fourier basis to provide the trajectory along the torus in phase space representing the biosignal cycle of the biosignal. As further described herein, projecting the measures of the biosignal onto the Fourier basis can include performing a principal component analysis (PCA) to transform the data onto a new coordinate system based on principal components. An example trajectory is further illustrated herein at least by FIG. 2B.

As further illustrated by FIG. 2B, the processor circuitry 102 can quantify the responsiveness of the biosystem to the perturbation based on clusters in the trajectory along the shape (e.g., torus). Clusters on the shape can capture biosystem behavior. When a new cluster forms, it can provide information about the phase in the biosystem. For example, new clusters can provide information about HRV for a cardiac rhythm for cardiac system behavior. As another example, a shift in a cluster can identify a change in amplitude of the biosignal, such as the heartbeat change in amplitude that can be caused by an issue (e.g., vasoconstriction). Further, the variance in the cluster (e.g., how far apart or close together the data points are and/or to the torus) or changes to the variance can provide information on the biosystem.

In some embodiments, the processor circuitry 102 can output an indication of homeostatic context based on a size and angle of the clusters. For example, the raw biosignals can be convolved (e.g., multiplied) with the principal components of the PCA to provide for clusters, and the size and angle of the clusters can be assessed. Example principal component can look like a sine wave at different frequencies. The sinusoidal appearance of the principal component suggests that there is an underlying cyclic structure in the data. The biosignal can be mapped to that cycle (whose frequency is f1) to gain insight into the behavior of the recorded biosignals. For the cardiac signal, this principal component can help define the shape (e.g., torus) and allows for quantifying phase lag in response to stimulation.

In some embodiments, the quantified responsiveness of the biosystem to the perturbation can be used to assess a biosystem state of the subject 102. For example, quantifying responsiveness of the biosystem to the perturbation can include identification of an effect of the biostimulation on vagal tone and/or changes to the biosystem from a baseline.

In some embodiments, the processor circuitry 102 can strobe the measures of the biosignal at a particular frequency associated with a feature of the biosignal. As used herein, strobing the measures of the biosignal includes and/or refers to the selection of a feature (e.g., R-spike or other peak or trough in the biosignal) and use of the features as a reference in the shape (e.g., torus) representation. The feature typically includes a peak or trough, but may include other frequency components (e.g., a dominant feature, frequency, and/or frequency of the principal component). To strobe the measures, the window timings can be advanced or retarded with respect to the frequency of the feature, which can slide the clusters in a clockwise or counter-clockwise direction. For example, for cardiac signals, the biosignal can be strobed at an R peak, such as 0.9 Hz. The R-R cycle can be mapped onto a circle, and the R spike is assigned angle zero on the torus. In some instances, the feature may be observed directly from the biosignal measures, such as with respiration. In other instances, the data is collected and analyzed using PCA, Fourier transforms, and other known techniques to pull out the frequency of the feature of the biosignal that can then be projected onto the shape (e.g., torus) to quantify periodic variations. Circadian rhythm and hormonal variation are other example biosignals that can be treated this way. The biostimulation can be applied and the effects observed for various signals, such as for quantifying changes in phase and amplitude of various hormones in response to biostimulation.

In some embodiments, the processor circuitry 102 can measure baseline biosignal measures and compare the baselines to the responses to the perturbation (e.g., biosignal measures in response to the plurality of biostimulation signals). The baseline measures can be received from the sensor circuitry 104 and prior to or otherwise without application of biostimulation to the target of the subject 101.

In various embodiments, the processor circuitry 102 can quantify responsiveness of the biosystem for the subject 102 over time. For example, the processor circuitry 102 can periodically cause the stimulation circuitry 106 to output the plurality of biostimulation signals (and/or variations thereof) and quantify the responsiveness over time using measures of the biosignal in response thereto. The periodic biostimulation output can be defined by and/or form part of a stimulus program, as further described herein.

In some embodiments, the responsiveness of the biosystem can be further based on feedback. The feedback can be intrinsic to the system 100, such as from the sensor circuitry 104, or extrinsic and communicated to the processor circuitry 102. The feedback can include additional measures of the biosignal, other physiological data, behavioral activity of the subject 101, environmental activity of the subject 101, and combinations thereof and/or other data.

In some embodiments, the processor circuitry 102 can cause the stimulation circuitry 106 to output an additional biostimulation signal in response to a biosystem state. For example, the additional biostimulation signal can form part of a stimulus program and/or can be an output of a machine learning model.

In various embodiments, the processor circuitry 102 includes or accesses a machine learning model which is trained using known inputs and known outputs, such as different biostimulation signals and measures of the biosignal. In some embodiments, the machine learning model is trained using the measures of the biosignal to identify a transfer pattern of stimulation parameters that optimize an effect associated with the biosignal and/or to predict a biosignal response and a homeostatic state. For example, the machine learning model can be trained using known biostimulation signals and biosignal responses to quantify responsiveness of the biosystem. The transfer pattern can be associated with different values of at least one stimulation parameter (e.g., pulse width) of the plurality of biostimulation signals and the effect on the biosignal. In some embodiments, the system 100 outputs an indication of the biosignal response and the homeostatic state (e.g., statis of homeostasis).

In some embodiments, the processor circuitry 102 can apply the trained machine learning model to the subject 101 or another subject to predict at least one of: (i) stimulation parameters that optimize the biosignal response or effect; and (ii) a homeostatic state. For example, the processor circuitry 102 can input the quantified responsiveness of the biosystem of the subject 101, the plurality of biostimulation signals, and/or the measures of the biosignal to the machine learning model to predict stimulation parameter(s) that improve tissue (e.g., vagus) tone and responsiveness. In some embodiments, in response, the processor circuitry 102 can cause the stimulation circuitry 106 to output an additional biostimulation signal with the predicted stimulation parameter(s).

As previously described, in some embodiments, the plurality of biostimulation signals include a plurality of neuromodulation signals applied to a nerve target of the subject 101, such as the vagus nerve or other nerve targets. In some embodiments, the target can include peripheral nerve, which is stimulated transcutaneously via the car, eyes, or head.

In various embodiments, the processor circuitry 102 can establish a stimulus program that causes the stimulation circuitry 106 to output an additional plurality of biostimulation signals to the subject 101 or other subjects and timing for the additional plurality of biostimulation signals to achieve a goal associated with the homeostatic state. The stimulus program can include a sequence of a plurality of biostimulation signals which can be guided by feedback from the sensor circuitry 104, an extrinsic data source, or a combination thereof.

In some embodiments, the biosignal can be used as a proxy for a second biosignal. In such embodiments, the processor circuitry 102 can include or have access to a machine learning model which is trained to: (i) identify a first transfer pattern that maps the measures of the second biosignal and the plurality of biostimulation signals;

and (ii) identify a second transfer pattern that maps the measures of the biosignal and the second biosignal. The processor circuitry 102 can input the biosignal, as a proxy for the second biosignal, to the machine learning model or a second machine learning model and to predict an effect of an additional biostimulation signal on the second biosignal. In some embodiments, the system 100 can further include second sensor circuitry configured to obtain measures of a second biosignal from the subject 101. In some such embodiments, the system 100 can output an indication of the predicted effect or cause output of the additional biostimulation signal to the target of the subject 101. As further described herein, the biosignal may be easier to capture using wearable or other technology than the second biosignal.

As described above, various embodiments involve the use and/or training of a machine learning model. Any of the above and below-described machine learning models can be trained by the processor circuitry 102 or other circuitry using an input dataset of known inputs and known outputs to identify a transfer pattern, among other applications.

In some embodiments, the processor circuitry 102 can train the machine learning model based on general population trends and demographic information associated with the subject 101. For example, the machine learning model can be trained using known inputs, such as demographically similar subjects (and/or the subject), and with the known outputs. Example known inputs include biostimulation signals including stimulation parameter(s) and/or biostimulation signal output, among other data. Example known outputs include biosignal measures or other physiological effects, responsiveness of the biosystem (e.g., clusters and variances in the torus, vagal tone), and/or homeostatic state. The input data used to train the machine learning model can include intrinsic and extrinsic data sources.

In some embodiments, the machine learning model is initially trained using demographic data and/or data of other subjects such that the machine learning model can be referred to as a demographic-machine learning model, which may not be specific to the subject 101 and/or may be a function of the particular demographic (e.g., age, sex, race, hereditary information). The demographic-machine learning model can be based on average trends for subjects of the particular demographic. The processor circuitry 102 can revise the demographic-machine learning model to be specific to the subject 101 to generate a subject-specific machine learning model based on data specific to the subject 101. This can include retraining or revising the machine learning model (e.g., the demographic machine learning model) using the subject-specific data. In some embodiments, the subject-specific data can include baseline biosignal measures and/or prior biosystem responses to perturbation. For example, the particular subject 101 can exhibit changes to the biosystem response to perturbation over time. By measuring overtime, the processor circuitry 102 can better predict changes to homeostatic state and/or issues with a biosystem.

Example demographic patterns for a subject can be based on age, sex, race, and/or other information, although embodiments are not so limited. In some embodiments, further demographic patterns can be learned by machine learning model over time, such as based on biosignals from a plurality of subjects.

As used herein, machine learning models can include data models which estimate or provide an output based on input data. Various machine learning frameworks are available from multiple providers which provide open-source machine learning datasets and tools to enable developers to design, train, validate, and deploy machine learning models, such as machine learning processors. Machine learning processors (sometimes referred to as hardware accelerators (MLAs), or Neural Processing Units (NPUs)) can accelerate processing of machine learning models. Machine learning processors are integrated circuits (ASICs) that can have multi-core designs and employ precision processing with optimized dataflow architectures and memory use to accelerate calculation and increase computational throughput when processing machine learning models.

Example machine learning models include artificial neural network, support vector machine (SVM), deep learning, cluster, and/or other models. An artificial neural network can estimate a function(s) that depends on inputs. In some embodiments, one or more layers of artificial neurons can receive input data and generate output data. Neural networks can include networks such as, but not limited to, learning networks (e.g., deep, deep structured, hierarchical, and the like), convolutional, auto-type networks (e.g., autoencoder, auto-associator), Diablo networks, and neural network models (e.g., feedforward, recurrent).

An SVM can utilize a linear classification. This classification can act to separate the data points into classes based on distance of the data points from a hyperplane. In some embodiments, the hyperplane is arranged to maximize the distances from the hyperplane to the nearest data points on either side of the hyperplane. This arrangement can group points located on opposite sides of the hyperplane into different classes. However, in some embodiments, the SVM can include a nonlinear classification that separates the data points with a hyperplane in a transformed feature space. The transformed feature space can be determined by one or more kernel functions, including nonlinear kernel functions. In some embodiments, the SVM is a multiclass SVM that separates data points into more than two classes, which can reduce a multiclass problem into multiple binary classification problems.

In some embodiments, a deep learning model can include models such as, but not limited to, convolutional networks (e.g., deep belief, neural), belief networks, Boltzmann machines, deep coding networks, stacked autoencoders, stacking networks (e.g., deep or tensor deep), hierarchical-deep models, deep kernel machines, and the like. It will be understood that such embodiments can include variants and/or combinations of the above-noted example networks.

In some embodiments, the machine learning model(s) can include a clustering method(s), which can include hierarchical clustering, k-means clustering, density-based clustering, among others. In some embodiments, the hierarchical clustering can be used to construct a hierarchy of clusters of the set of features. In some embodiments, the hierarchical clustering utilizes a “bottom up” approach (e.g., agglomerative) wherein each data point starts in its own cluster, and pairs of clusters are merged at progressively higher levels of the hierarchy. However, in some embodiments, the hierarchical clustering utilizes a top-down approach in which all data points start in one cluster, and then clusters are split at progressively lower levels of the hierarchy.

In some embodiments, the k-means clustering implementation can include placing the set of features into k clusters, where k is an integer equal or greater than two. Via such clustering, each data point belongs to a cluster having a mean that is closer to the data point than any means of the other clusters. However, in some embodiments, a machine learning model can include density-based clustering, which can be used to group together data points that are close to one another, while identifying as outliers any data points that are far away from other data points.

In some embodiments, a machine learning model can include a mean-shift analysis that can be used to determine the maxima of a density function based on discrete data sampled from that function.

In some embodiments, a machine learning model can include structured prediction techniques and/or structured learning techniques. Such techniques can be used to predict structured objects and/or structured data, such as structured sets of features and/or sensor data. In some embodiments, such structured prediction and/or structured learning techniques can include graphical models, probabilistic graphical models, sequence labeling, conditional random fields, parsing, collective classification, bipartite matching, Bayesian networks or models, and the like. It will be understood that such embodiments include variants and/or combinations of the above-noted example techniques.

Machine learning can be useful as biosystems may not be linear. Stimulation must penetrate tissue and the biostimulation waveform at the target may not be the waveform emitted by the stimulation circuitry 106 (e.g., an electrode). For example, the waveform emitted by the stimulation circuitry 106 can be transformed when penetrating through tissue to the target of the subject 101, which is complex. Further, various physiological effects, such as activation of brain patterns, can be difficult to measure directly and can be linked to a second biosignal that is easier to sense.

FIGS. 2A-2B illustrate example biostimulation signals and measures of a biosignal responsive thereto, in accordance with the present disclosure. As shown by FIG. 2A, the plurality of biostimulation signals can be represented as a waveform file 220, with each epoch 222-1, 222-2, 222-3, 222-4 corresponding to a different value for a stimulation parameter. The waveform file 220 can be stored on a depository, as previously described. In the example waveform file 220, there are four epochs 222-1, 222-2, 222-3, 222-4 with washout periods of no-stimulation between. Each region of an epoch, such as the region 224 of epoch 222-2, includes pulses of stimulation for a period of time followed by non-stimulation for the period of time. For example, the epochs 222-1, 222-2, 222-3, 222-4 can include three regions of pulsed stimulation for sixty seconds followed by non-stimulation for sixty seconds for a total epoch time of five minutes, and washout periods of five minutes in which stimulation is not applied the entire time. Embodiments are not so limited and can include other periods of time for the epochs and the washout periods, as well as other waveform shapes.

Various embodiments process the data by performing a PCA. PCA is a known linear dimensionality reduction technique that linearly transforms data onto a new coordinate system of principal components which capture the largest variation in the data. The principal components are a sequence of p unit vectors of a collection of points in a real coordinate space, wherein the i-th vectors is the direction of a line that best first the data while being orthogonal to the first i−1 vectors. For example, the first principal component of a set of p variables is the variable formed as a liner combination of the original variables that explains the most variance. The second principal component explains the most variance in what is left once the effect of the first principal component is removed. This process can proceed through all p iterations, until all the variance is explained. The first principal component can equivalently be defined as a direction that maximizes the variance of the projected data.

FIG. 2B illustrates an example projection 226 of biosignal measures onto a shape, such as the illustrated torus 228. As previously described, biosignal measures can be obtained over sample windows and responsive to a plurality of biostimulation signals having different values for a stimulation parameter. The biosignal measures are strobed at a frequency of a feature, such as an R peak, by centering at the frequency and time working to the R peak at either end of the time interval. The data can be low-pass filtered with a frequency cut-off and then a PCA can be performed over the windows, with the first three principal components plotted as illustrated by component 0, component 1, and component 2. The different shapes 223-1, 223-2, 223-3, 223-4 (as shown by FIG. 2A) represent the different values for the stimulation parameter. For example, the plurality of biostimulation signals can include signals applied at four different pulse widths, and each are plotted.

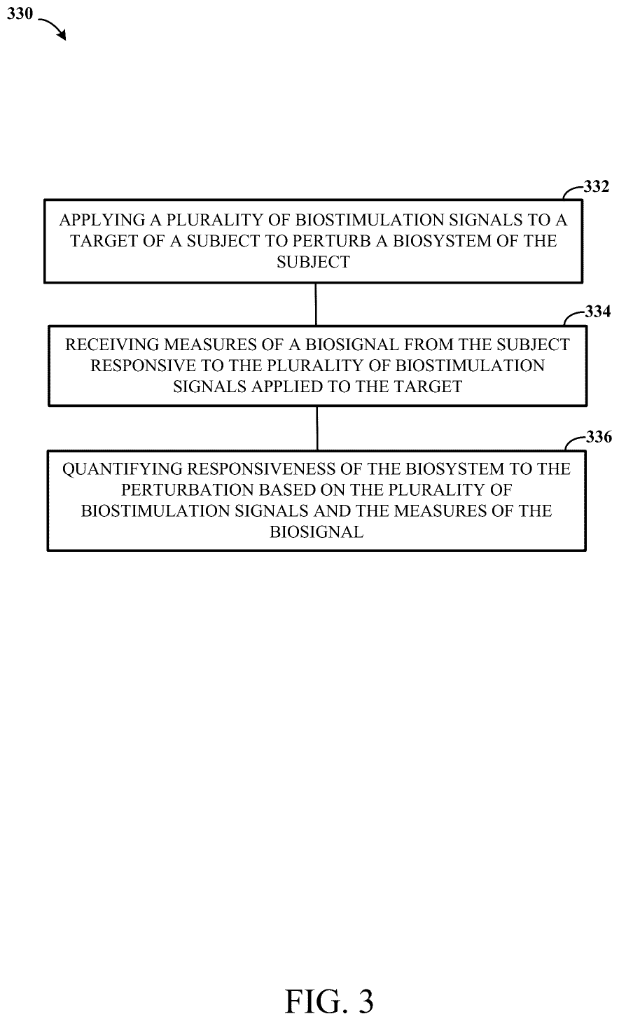

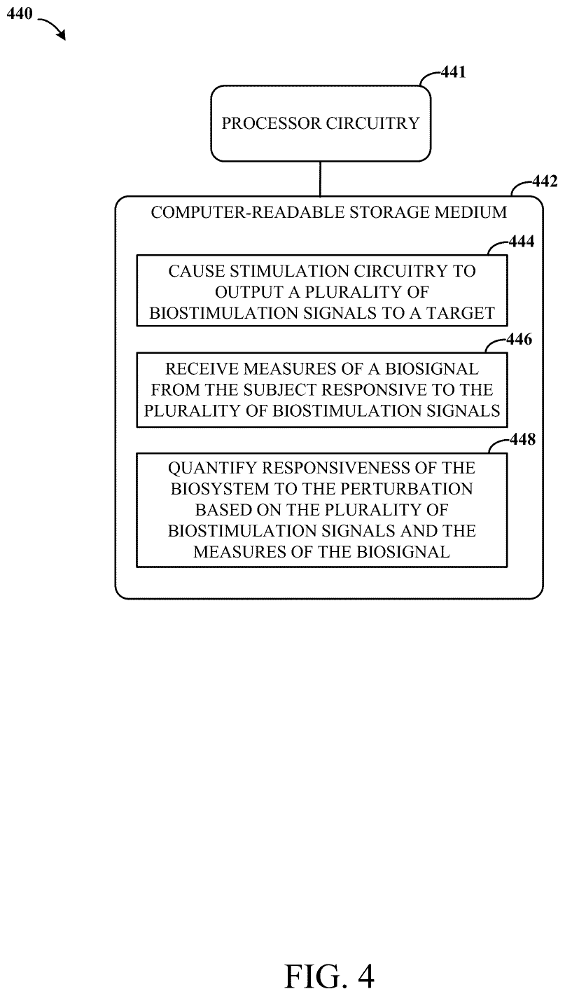

FIG. 3 illustrates an example method for quantifying responsiveness of a biosystem of a subject, in accordance with the present disclosure. The method 330 can be implemented by the system 100 of FIG. 1 and/or the computing device 440 of FIG. 4.

At 332, the method 330 includes applying a plurality of biostimulation signals to a target of a subject to perturb a biosystem of the subject, and at 334, receiving measures of a biosignal from the subject responsive to the plurality of biostimulation signals applied to the target. In various embodiments, as previously described, the plurality of biostimulation signals can be downloaded from external memory circuitry, wherein each of the plurality of biostimulation signals represent a processed tissue signal as a sequence of at least one state corresponding to a set of state parameters and correlated with causing a particular physiological effect of perturbing the biosystem. In some embodiments, the downloaded plurality of biostimulation signals can be stored locally on a depository.

At 336, the method 330 includes quantifying responsiveness of the biosystem to the perturbation based on the plurality of biostimulation signals and the measures of the biosignal. As previously described, quantifying the responsiveness of the biosystem can includes providing a trajectory of the measures of the biosignal along a shape in phase space representing a biosignal cycle of the biosignal, and quantifying the responsiveness of the biosystem to the perturbation based on clusters in the trajectory along the shape. For example, the method 330 can include providing the trajectory along a torus in phase space representing the biosignal cycle of the biosignal by projecting the measures of the biosignal onto a Fourier basis, and quantifying the responsiveness of the biosystem to the perturbation based on clusters in the trajectory along the torus.

In some embodiments, the method 330 includes receiving baseline measures of the biosignal without biostimulation applied and comparing the baselines measures to the measures of the biosignal responsive to the plurality of biostimulation signals applied to the target to quantify the responsiveness of the biosystem.

In some embodiments, the method 330 includes identifying a transfer pattern of stimulation parameters that optimize an effect associated with the biosignal and/or the biostimulation using a machine learning model which is trained using the plurality of biostimulation signals and the measures of the biosignal. In some embodiments, the method can include outputting an indication of the stimulation parameters that optimize the effect or outputting an additional biostimulation signal to the target at the optimized stimulation parameters.

As noted above, the effect can include a physiological effect. As used herein, a physiological effect includes and/or refers to a change or response in the body which can be measured, such as via biosignals or environmental state signals. Example physiological effects include changes in biosignal measures or maintaining particular values, homeostatic states or setpoints, among others. Embodiments can further include other types of effects, such as behavioral effects. A behavioral effect includes and/or refers to a change or response to behavior of the subject, and which may not be measured or may be difficult to measure directly. In some embodiments, the effects can include combinations of physiological effects and behavioral effects.

In some embodiments, the method 330 includes receiving measures of a second biosignal from the subject, and using a machine learning model to: (i) identify a first transfer pattern that maps the measures of the second biosignal and the plurality of biostimulation signals, and (ii) identify a second transfer pattern that maps the measures of the biosignal and the second biosignal. The biosignal may be easier to detect and/or may be more accurate than the second biosignal. For example, the biosignal can include a cardiac signal which can be captured using wearable devices and the second biosignal can include neural responses which are captured using MRI data. In some such embodiments, the method 330 further includes inputting the biosignal, as a proxy for the second biosignal, to the machine learning model or a second machine learning model and to predict an effect of an additional biostimulation signal on the second biosignal.

The method 330 can include further variations and additional features, such as those described above in connection with the system 100 of FIG. 1. For example, the method 330 can include any of the features executed by the processor circuitry 102, the sensor circuitry 104, and/or the stimulation circuitry 106. The common features and attributes are not repeated for each of reference.

FIG. 4 illustrates an example computing device including non-transitory computer-readable storage medium storing executable instructions, in accordance with the present disclosure. In some embodiments, the processor circuitry 441 can form part of a computing device 440. The computing device 440 includes the processor circuitry 441 and computer-readable storage medium 442 storing a set of instructions 444, 446, 448. Although embodiments are not so limited, and the processor circuitry 441 and computer-readable storage medium 442 can form a part of distributed computing devices, with the distributed computing devices being in communication with each other. The computer-readable storage medium 442 can, for example, include read-only memory (ROM), random-access memory (RAM), electrically erasable programmable read-only memory (EEPROM), Flash memory, a solid state drive, and/or discrete data register sets. As used herein, “executable instructions” can be interchanged with “instructions executed by the processor circuitry”. In some embodiments, any of the computing device 440, the processor circuitry 441, and computer-readable storage medium 442 can respectively include an implementation of and/or at least some of substantially the same features and attributes as the computing device 108, the processor circuitry 102, and the memory circuitry 110 of FIG. 1.

At 444, the processor circuitry 441 can execute instructions to cause stimulation circuitry to output a plurality of biostimulation signals to a target of a subject to perturb a biosystem of the subject. At 446, the processor circuitry 441 can execute instructions to receive measures of a biosignal from the subject responsive to the plurality of biostimulation signals. At 448, the processor circuitry 441 can execute instructions to quantify responsiveness of the biosystem to the perturbation based on the plurality of biostimulation signals and the measures of the biosignal responsive to the plurality of biostimulation signals.

Embodiments are not limited to the instructions illustrated by FIG. 4. In some embodiments, additional or alternative instructions can be stored. For example, the processor circuitry 441 can execute instructions to receive measures of a biosignal from sensor circuitry in response to a plurality of biostimulation signals output to a target of a subject and train a machine learning model, using the measures of the biosignal and stimulation parameters of the plurality of biostimulation signals, to identify a transfer pattern of the biostimulation signals and the biosignal. For example, the instructions to train the machine learning model can include instructions executable to collect data from a plurality of subjects including measures of a plurality of biosignals and stimulation parameters of a plurality of biostimulation signals as applied to the plurality of subjects and to train the machine learning model using the collected data. In some embodiments, the processor circuitry 441 can apply the trained machine learning model to predict an effect of a particular biostimulation signal output to the target. The effect can include a physiological effect, such as changes to the biosignal measure and/or impacts on a biosystem, among others. In some embodiments, the processor circuitry 441 can further to apply the trained machine learning model to predict a biostimulation signal to output to the target to cause a particular effect.

The instructions can include further variations and additional features, such as those described above in connection with the system 100 of FIG. 1. For example, the processor circuitry 441 can execute instructions to implement any of the features executed by the processor circuitry 102 of FIG. 1. The common features and attributes are not repeated for each of reference.

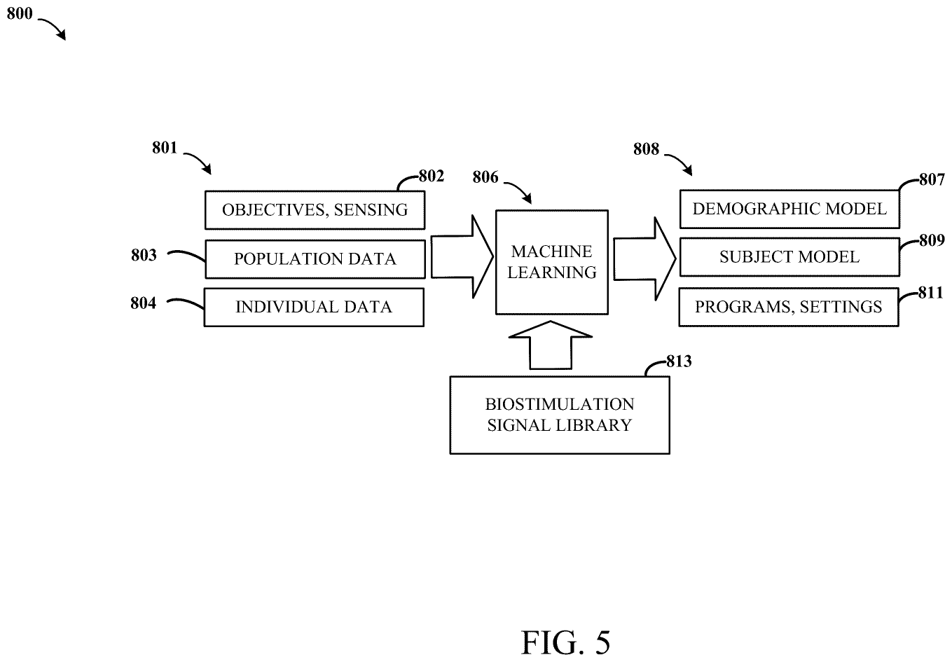

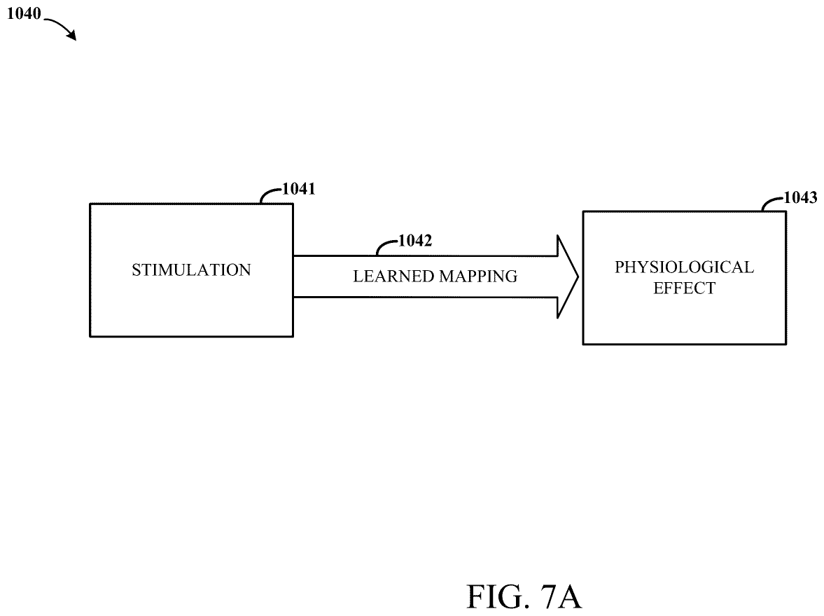

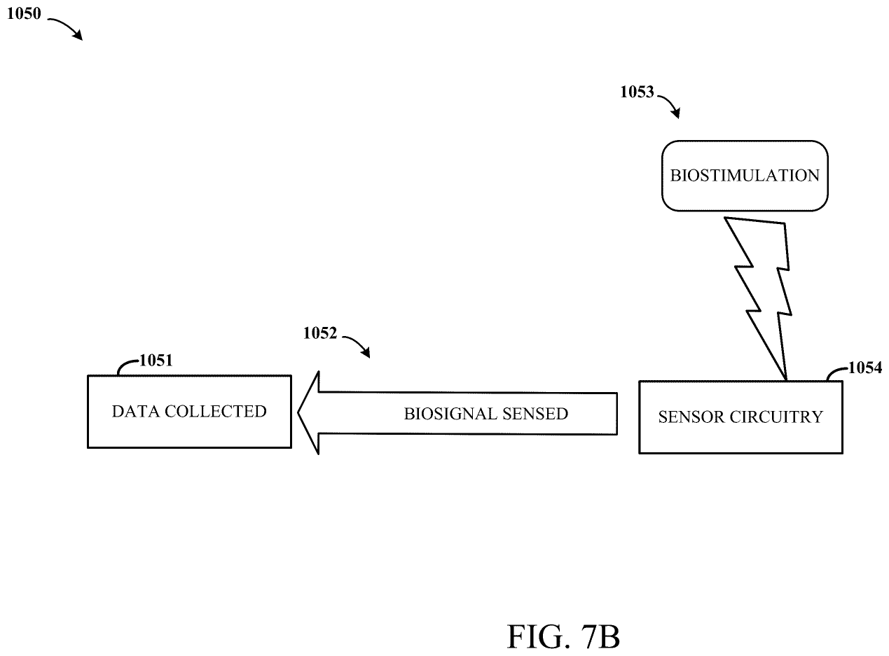

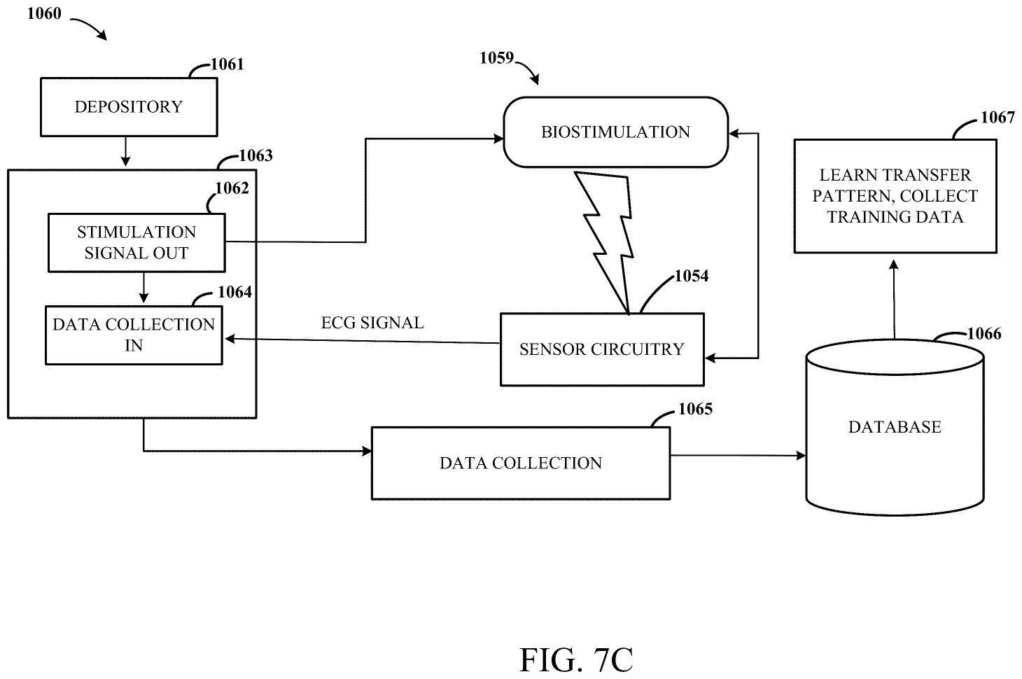

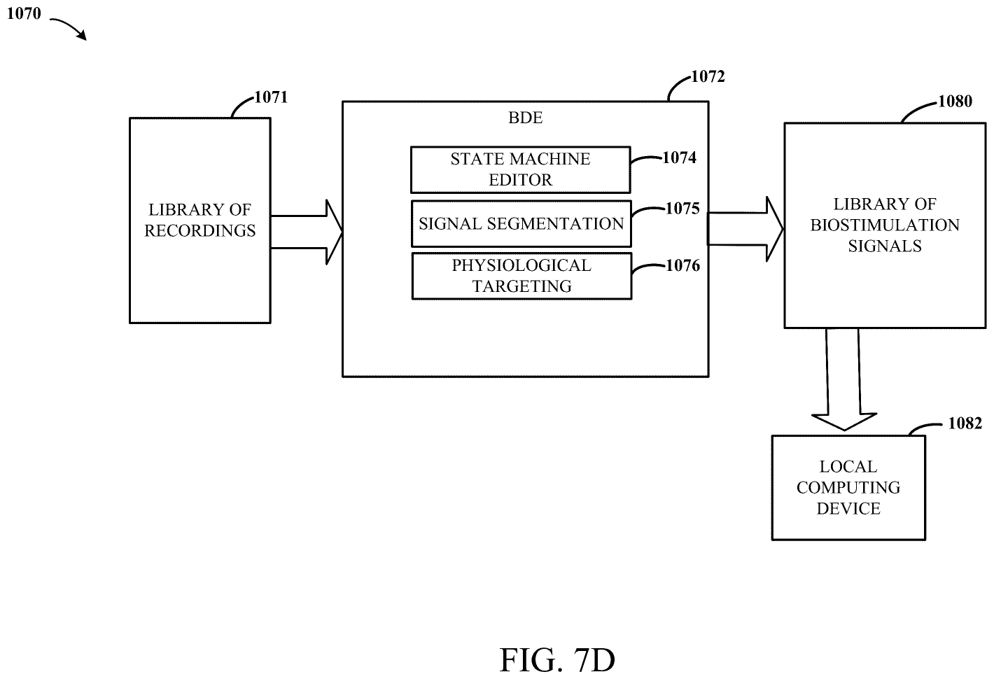

FIG. 5 illustrates an example machine learning process, in accordance with the present disclosure. More particularly, FIG. 5 shows a schematic view of an example machine learning process 800, which can be used to train different machine learning models 808. For example, different types of data sets 801 can be input for machine learning which include known inputs and known outputs for training different machine learning models 808. The data sets 801 can include objectives and sensing 802, demographic data 803 including sensed signals and biostimulation signals, individual data 804 including sensed signals and biostimulation signals. As previously described, the sensed signals can include biosignals which are derived from the biological state and signals derived from the environmental data. The objective can be specific effect or set of effects (e.g., goals) for the subject and/or homeostasis objective. The data set(s) 801 can be input to machine learning layer 806 for learning from the recorded and marked data of demographic subjects for generating the demographic-machine learning model 807 and then fine-tuned to generate a subject-specific machine learning model 809. Based on objectives input (e.g., 802), the model(s) 807 and/or 809 can be updated to generate a stimulus program 811. For example, the stimulus program 811 can contain recorded neurograms and/or other tissue signals, synthesized neuromodulation signals and/or other biostimulation signals, scheduled timings, and stimulation parameter values. As previously described, a set of different biostimulation signals can be stored as a library 813, and subset associated with a particular effect(s) can be downloaded from the library 813 and stored locally on a depository. Different example machine learning models are further illustrated and described in connection with FIGS. 7A-7D.

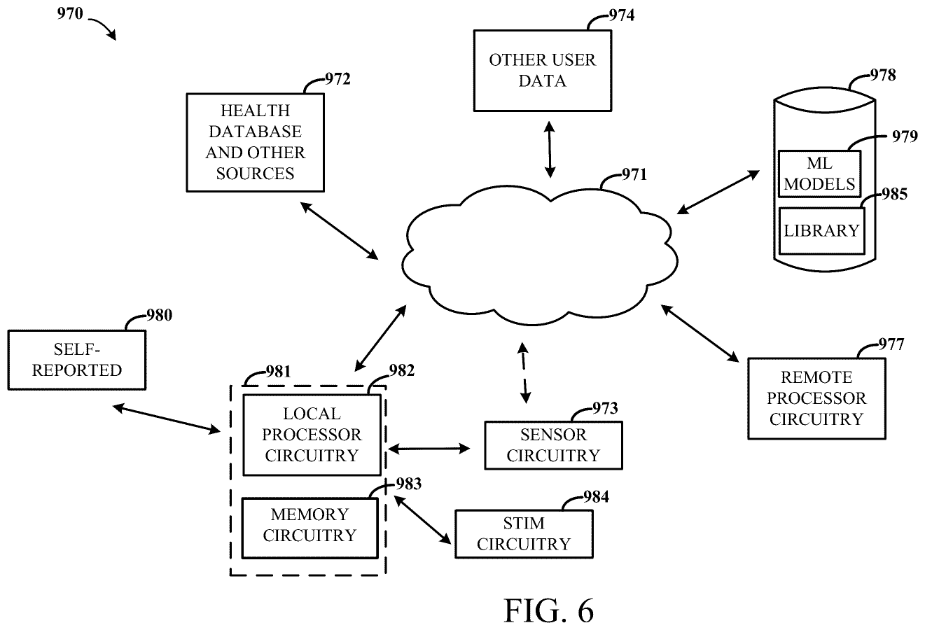

FIG. 6 illustrates an example system including machine learning models, in accordance with the present disclosure. As shown, a variety of computing resources 973, 974, 977, 978, 982, 983, 984 can form the system 970. In some embodiments, the system 970 can include a cloud computing system or other type of network system which includes a plurality of distributed computing resources. The distributed computing resources can be at different locations, such as local computing resources 982, 983, 973 which are located proximal to the subject and remotely-located computing resources 977, 978 which are located remotely from the subject. The various computing resources 973, 974, 977, 978, 982, 983, 984 can communicate with one another using data communications over a network 971. For example, the system 970 can be cloud-based and/or can be implemented through other computer systems in alternative architectures, such as a peer-to-peer network.

The system 970 includes a local processor circuitry 982 and local memory circuitry 983, and which can form part of a computing device 981. The computing device 981 can be local to the subject, such as a smartphone, laptop, desk computer, or other device that is accessible to the subject. The computing device 981 can be in communication with stimulation circuitry 984 and/or sensor circuitry 973 that is local to the subject. The memory circuitry 983 can store instructions executable by the processor circuitry 982. In some embodiments, the memory circuitry 983 can store a local version of a machine learning model or models 979 and/or portions of a library 985 which can be obtained from a back-end database 978. In some embodiments, the machine learning model or models 979 and/or portions of a library 985 can be stored temporarily on the local processor circuitry 982 (e.g., in cache memory).

In some embodiments, the machine learning model 979 is stored on the back-end database 978 and processed remotely by a remotely-located processor circuitry 977. Although one database 978 is illustrated, the system 970 can include a plurality of databases stored on memory circuits which are accessible by a plurality of distributed processor circuits which can train the machine learning models 979. For example, the remotely-located processor circuitry 977 can construct and train the machine learning model 979 and provide the trained machine learning model 979 to the local processor circuitry 982. In some such embodiments, either the local processor circuitry 982 or the remotely-located processor circuitry 977 can revise (e.g., retrain) the machine learning model 979 and/or biostimulation signals using tracked sensor data from the sensor circuitry 973 or other feedback data, as previously described. In other embodiments, the remotely-located processor circuitry 977 can remotely apply the trained machine learning model 979 to input data, which is communicated to the remotely-located processor circuitry 977 directly from the sensor circuitry 973 or from the local processor circuitry 982.

The system 970 can include other inputs which can be used to generate the machine learning models 979 and/or revise the trained machine learning models 979 to be subject-specific. In some embodiments, the additional inputs include self-reported measures 980, such as subject provided inputs on a condition. The self-reported measure 980 can be communicated via input circuitry to the local computing device 981. In some embodiments, the system 970 can include health databases or other sources of health information 972, such as a health application or patient portal to a professional and which allows for feedback information or self-reported measures to be input as a features to the machine learning models 979.

FIGS. 7A-7D illustrate example machine learning models, in accordance with the present disclosure. The various machine learning models can be generated and/or trained locally by local processor circuitry and/or remotely and stored on a database, such as illustrated by FIG. 6.

FIG. 7A illustrates a schematic of an example machine learning model for stimulation which can be associated with perturbing a biosystem. In various embodiments, a machine learning model 1040 can be trained to learn a transfer pattern that maps biostimulation signals 1041 applied and the effect 1043, as shown by 1042. As previously described, a plurality of biostimulation signals with different values for a stimulation parameter(s) can have different effects. For example, the stimulus frequency and amplitude for neuromodulation, such as VNS, can result in the excitation of different groups of nerve fibers, depending on their electrical properties. The transfer pattern can be used as a model of a nerve and/or other tissue used to predict the effects of stimulation. In particular, the machine learning model 1040 can associate stimulation patterns by training on a combination of collected data and in silico stimulation. The machine learning model 1040 can be trained to map stimulation patterns to effects or conversely, map effects to stimulation patterns.

FIG. 7B illustrates an example machine learning model for sensing. In various embodiments, a machine learning model can be trained to learn a transfer pattern that maps observable variables with latent variables which are difficult to observe in normal usage. For example, observable variables can include data that is readily accessible, such as a biosignal sensed 1052 through wearable devices (e.g., chest strap or smartwatch). An example observable biosignal includes ECG. In contrast, MRI data is difficult to obtain, but provides a map of brain activity during stimulation. This is an example of a latent variable. Embodiments are not limited to biosignals, and variables can include extrinsic observables, such as skill-learning visual cues.

As a specific example, machine learning can be used to learn the transfer pattern between ECG data and fMRI-derived brain activity that allows brain activity to be inferred during normal usage. This allows the cardiac signal to be used as a proxy signal for brain activity. The machine learning model 1050 is trained and validated using data from controlled and fixtured experiments that, while difficult to perform, need only be executed during a setup phase for training. This application of machine learning can be used to associate any easily observed set of observables with latent variables. For example, once trained, sensor circuitry 1054 can be used to sense ECG data (e.g., 1052) responsive to neuromodulation signals output 1053 and use the ECG data as a proxy for brain activity as the data collected 1051.

FIG. 7C illustrates an example implementation of a machine learning process to enable control of a cardiac signal through breathing. As shown, a machine learning process can involve a system 1060 including a computing device 1063 which includes or has access to a depository 1061 of biostimulation signals and outputs biostimulation signals, in a digital form, from the depository 1061 to stimulation circuitry, as shown at 1062. The stimulation circuitry converts the biostimulation signal to analog form and outputs the biostimulation signal to a target of the subject, at 1059. Sensor circuitry 1054 obtains measures of a biosignal in response to the biostimulation signal, such as the illustrated ECG, and provides the biosignal measures to the computing device 1063, as illustrated at 1065. The collected data is output to a database 1066, as illustrated at 1065. The database 1066 can include identification of the biostimulation signals applied, the biosignal measures, and time-stamped event data. Using the database 1066, transfer patterns can be learned between stimulation patterns and homeostatic responses, such as stimulation patterns associated with changes in homeostasis, as illustrated at 1067. The training data can be collected to learn stimulation patterns associated with different disease states or other biosystem states. For example, the input data set can include stimulation patterns and homeostatic responses from subject(s) known to be at the different disease states, homeostasis setpoints, or other types of biosystem states. In some embodiments, the input data (e.g., training data) can be collected to learn associations between stimulation patterns and an objective function, such as a goal, response optimization, or other effect.

In some embodiments, the objective function can include generating a demographic-machine learning model that is standardized across many individuals. For example, the machine learning model can be trained to associate biostimulation with responses in a subject-agnostic manner. The machine learning model can be trained using stimulation-sensing measurements across a population to learn transfer patterns that map from stimulation to effect and create subject-agnostic stimulus programs for optimizing an effect.

In some embodiments, a machine learning model can be trained for modeling time-varying homeostasis. Homeostasis is generally not static but is a dynamic equilibrium, where the homeostasis state can change over time with diurnal variation, aging, and inflamed/not inflamed. Homeostasis setpoints include and/or refer to physiological values of biosignals or other targets which are the center of or within a threshold range of values. The threshold range of values may be considered to be healthy and/or normal for a subject. Homeostasis setpoint targets can be trajectories, including for example: (i) body temperature (e.g., 37 degrees Celsius), (ii) serum levels of glucose, cytokines, and (iii) cardiac state (e.g., HR, HRV). Trajectories can be target effects, but the control problem is complex and can be approached using machine learning models that are appropriate for temporal sequences. For example, the machine learning model can be trained to predict, given a homeostasis trajectory, what stimuli to apply to achieve a homeostasis setpoint.

The different machine learning models can individually or together be used to predict the effects of biostimulation, learn mappings of observable variable(s) to latent variables, and/or learn mappings of control time-varying homeostasis using a stimulus program or programs that include a sequence of biostimulation guided by feedback from intrinsic and extrinsic sensing. For example, the machine learning models can learn the mappings between stimulation and responses that are individualized, learn associations between responses to perturbations and disease state or desired homeostasis setpoints, learn stimulation sequences that optimize objective functions around sensed responses, and standardized these mappings across a demographic, among other techniques.

FIG. 7D illustrates an example machine learning system 1070 which can be used to optimize a particular effect, such as for homeostatic state.

The system 1070 includes a library of recordings 1071, such a tissue signals. A tissue signal can be an electrical or other recording representing of the state of the tissue. A tissue signal can be processed, e.g., by a state machine, to generate synthetic biostimulation signals of a processed tissue signal. In some embodiments, the tissue signal can include a neurogram which is processed to generate a synthetic neuromodulation signal. A state machine representation or representation can include and/or refer to a mathematical model or a numerical model of a stimulus (e.g., a computation model defined by sets of states, initial states and inputs/causes of transitions between states). In some embodiments, each state in the state machine representation corresponds to a set of state parameters that dictate a known spike amplitude and timing interval. For example, a processed tissue signal can have an associated set of synthetic biostimulation signals, such that application of the stimulus according to the set of state parameters can result in the known or expected spike amplitude and timing.

In some embodiments, a biostimulation development environment (BDE) 1072 can be used to assemble biostimulation signals from experimental and synthetic sources. The library of recordings 1071 can be input to the BDE 1072 to output synthetic biostimulation signals 1080. For example, neurograms include structures spikes that represent recruitment of nerve fibers to inform caudal (post-brainstem) targets about non-motor and non-sensory somatic state. As previously described, tissue signals, such as neurograms, can be characterized in part by the evolution of firing rate and amplitude within the spike train. Different stimuli appear to give rise to distinct tissue signal structures. The library of recordings 1071 can include tissue signals recorded after placing a subject under a particular condition. The structures of different tissue signals can be used to build parameterized state-machine models of synthetic biostimulation signals to evoke particular, specific responses in a subject. The synthetic biostimulation signals can be modified by changing the state parameters which were used to create the synthetic biostimulation signal.

As previously described, each state in the state-machine model can define a set of state parameters that can be used to stochastically, or deterministically, generate biostimulation signals (BMSs) in the form of a spike train of desired amplitude and rate. A BMS can be defined by a sequence of states that identify distribution parameters and duration for spike train generation at each state, along with state transitions that define duration and state-to-state interpolation methods. New biostimulation signals can be generated at will without the need for surgery, recording, or sacrifice of animal subjects, such as for neuromodulation signals as described in U.S. Pat. No. 11,752,233. For example, biostimulation is applied to elicit an intended effect and tissue signals are recorded in response. The tissue signals are digitized by the BDE 1072 and processed using a state machine to generate the state-machine representations of the tissue signals which include the at least one state parameter and can be used to generate the biostimulation signals. The state machine editor 1074 can edit the state-machine representation based on signal segmentation 1075 and/or physiological targeting 1076. Physiological targeting includes and/or refers to the spatial effects, or possibility thereof, arising from the choice of waveform. One example is focused ultrasound to selectively stimulate tissue. Another example is the use of specific frequency and power combinations to achieve a desired penetration depth for stimulation. The segmentation 1075 can include hand segmentation, where a user identifies state intervals, or states and segmentations are learned by a machine learning model. After segmentation, the state machine editor 1074 can compute state parameters and save the state machine descriptors. In some embodiments, a machine learning model can learn from prior data about different states and can include probabilities of a spike belonging with that state. The machine learning model can be a Markov model that learns hidden states or variables for state and/or effect, such as for physiological targeting.

The synthetic neuromodulation signals 1080 can be provided as a library from which a local computing device 1082 can selectively download a plurality of neuromodulation signals for a particular effect.

As used herein, a neurogram includes and/or refers to a measurement of the signals that traverse a nerve. In one embodiment, the neurogram may be produced in response to application of a particular condition of a subject. It should be realized that use of the term “subject”, as used herein, includes any animal or human subject that is put under a condition and provides a neurogram and/or other tissue signals. For example, the condition may be wherein the subject has been given a particular treatment, such as by administration of a drug. One example of a neurogram includes a structured sequence of electrical neuronal spikes, where the sequence of electrical neuronal spikes has a characteristic amplitude envelope, an inter-spike interval profile and a definite extent in time (e.g., a defined time interval). A neurogram can be an electrical recording representing of the state of a peripheral nerve. A neurogram can be processed by, e.g., a finite state machine to generate synthetic neuromodulation signals of a processed neurogram.

As used herein, state machine representation or finite state machine representation may refer to, e.g., a mathematical model or a numerical model of a stimulus. In some embodiments, each state in the state machine representation corresponds to a set of state parameters that dictate a known spike amplitude and timing interval. For example, a processed neurogram can have an associated set of synthetic neuromodulation signals, such that application of the stimulus according to the set of parameters can result in the known or expected spike amplitude and timing.

The skilled artisan would recognize that various terminology as used in the Specification (including claims) connote a plain meaning in the art unless otherwise indicated. As examples, the Specification describes and/or illustrates aspects useful for implementing the claimed disclosure by way of various circuits or circuitry which may be illustrated as or using terms such as blocks, modules, device, system, unit, controller, and/or other circuit-type depictions. Such circuits or circuitry are used together with other elements to exemplify how certain embodiments may be carried out in the form or structures, steps, functions, operations, activities, etc. For example, in certain of the above-discussed embodiments, one or more modules are discrete logic circuits or programmable logic circuits configured and arranged for implementing these operations/activities, as may be carried out in the approaches shown in FIG. 1. In certain embodiments, such a programmable circuit is one or more computer circuits, including memory circuitry for storing and accessing a program to be executed as a set (or sets) of instructions (and/or to be used as configuration data to define how the programmable circuit is to perform), and an algorithm or process as described at FIG. 4 is used by the programmable circuit to perform the related steps, functions, operations, activities, etc. Depending on the application, the instructions (and/or configuration data) can be configured for implementation in logic circuitry, with the instructions (whether characterized in the form of object code, firmware or software) stored in and accessible from a memory (circuit).

Various embodiments are implemented in accordance with the underlying Provisional Application No. 63/534,058, filed Aug. 22, 2023, and entitled “Systems and Methods for Neural Sounding for Homeostatic Health”, to which benefit is claimed and which is fully incorporated herein by reference in its entirety for its general and specific teachings. For instance, embodiments herein and/or in the Provisional Application can be combined in varying degrees (including wholly). Reference can also be made to the experimental teachings and underlying references provided in the underlying Provisional Application. Embodiments discussed in the Provisional Application are not intended, in any way, to be limiting to the overall technical disclosure, or to any part of the claimed disclosure unless specifically noted.

### EXPERIMENTAL EMBODIMENTS

Various experimental embodiments were directed to perturbing a biosystem of a subject and quantifying responsiveness of the biosystem in response thereto. More specifically, various experimental embodiments were directed to applying a plurality of biostimulation signals to a subject to perturb a biosystem of the subject and observe responses thereto to quantify responsiveness of the biosystem and assess for homeostatic health.

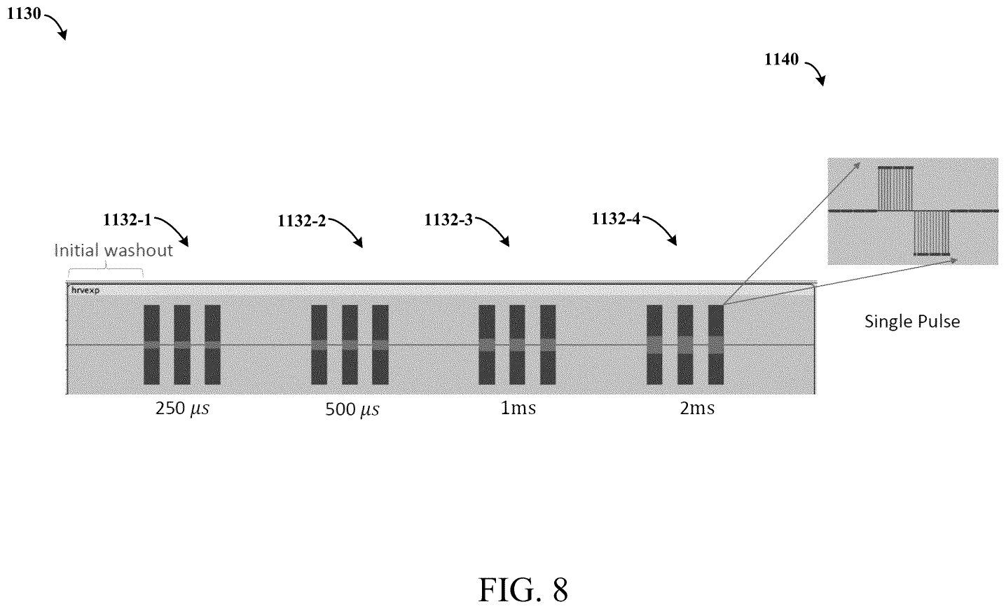

FIG. 8 illustrates an example of a plurality of biostimulation signals used to perturb a biosystem of a subject, in accordance with the present disclosure. In specific embodiments, electrical stimulation was applied at the tragus, which was ground at the earlobe, using an earpiece with the amplifier gain set to 22 and using a biphasic wave pulse at 25 Hz. The stimulation was represented as a waveform file 1130 and had four epochs of stimulation 1132-1, 1132-2, 1132-3, 1132-4 with a washout period (e.g., no stimulation) of five minutes before the stimulation and after each epoch 1132-1, 1132-2, 1132-3, 1132-4. Each epoch of stimulation 1132-1, 1132-2, 1132-3, 1132-4 included three repetitions of sixty seconds of pulsed stimulation and sixty seconds of no stimulation, as shown by the single pulse 1140 which includes biphasic wave pulses at 25 Hz. The epochs of stimulation 1132-1, 1132-2, 1132-3, 1132-4 respectively included different values for the pulse width, including a pulse width of 250 microseconds (μs), 500 μs, 1 milliseconds (ms), and 2 ms.

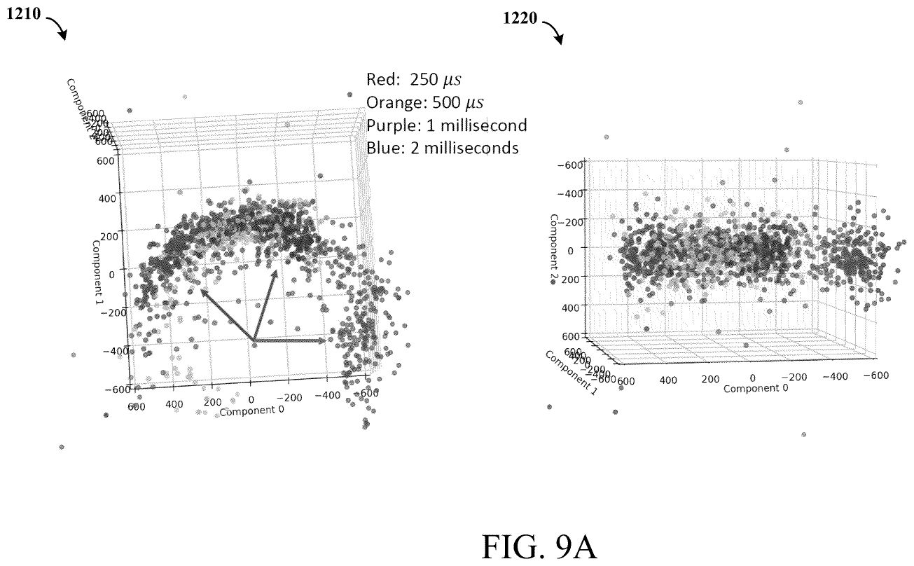

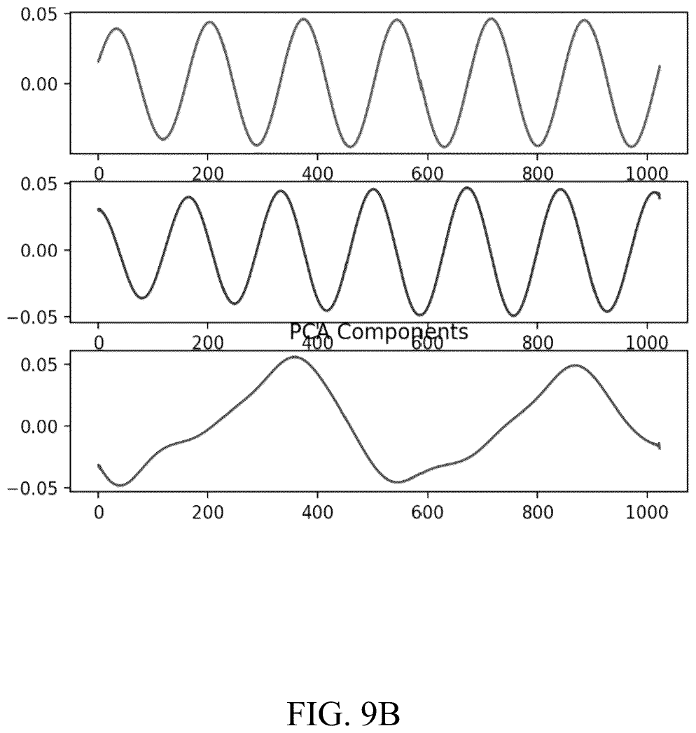

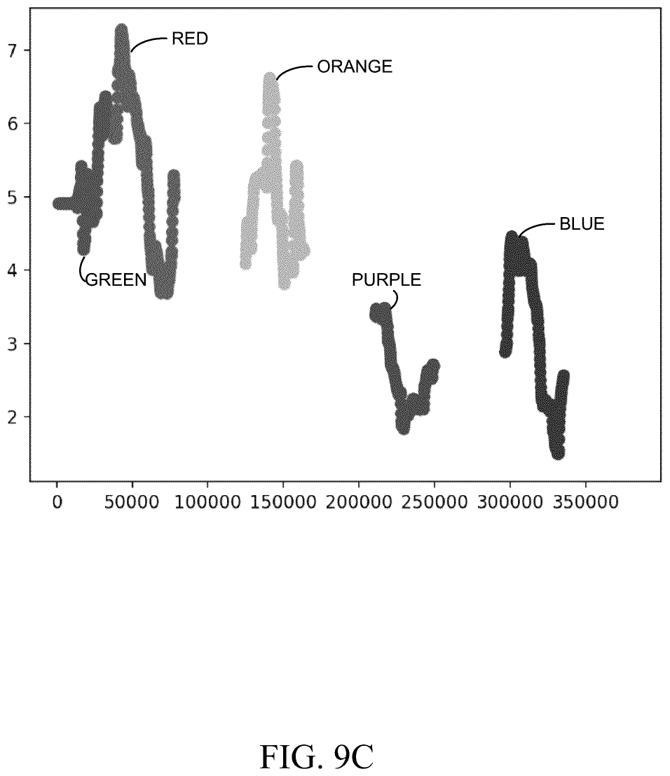

FIGS. 9A-9C illustrate example biosignal response to the plurality of biostimulation signals of FIG. 8, in accordance with the present disclosure. The biosignal of ECG was measured in response to applying the plurality of biostimulation signals. ECG was sampled over 512 windows as centered at the R peak, with 5 peaks totals and time warped to the peaks at either end of the interval. The data was labeled using colors by the stimulation intervals which correspond to the different pulse widths and with the washout periods not displayed. The data was low-pass filtered with a cut-off at 1.5 Hz and then PCA was performed over the windows with the first three principal components plotted. FIG. 9A includes graphs 1210, 1220 of the resulting three-dimensional (3D) PCA scatter plots. FIG. 9B includes graphs of the first three principal components. As shown, the first two principal components (e.g., top and middle graph) are sinusoids. With sinusoids, data points will map to a circle or an arc and the fundamental frequency is f1. The first two principal components represented around a 0.8 Hz passband in the frequency domain and the third principal component was greater than 0.8 Hz passband. The biostimulation appeared to affect phase angle and cluster variances. The cluster variance was believed to be correlated with HRV. The third principal component was believed to be correlated with respiration. FIG. 9C includes a graph of HRV with the X axis being time (in samples) and Y axis being an L1-norm HRV measure. HRV radius is 32 beats, with 64 beats per HRV point. The biostimulation signals applied at a pulse width of 250 μs (red data points) and biostimulation signals applied at a pulse width of 500 μs (orange data points) showed high HRV, suggesting that HRV decreases with increased pulse width.

In any of the graphs of 3D PCA scatter plots illustrated herein, including those in FIGS. 9A, 10, 11, 12, and 13, one of the axis may be pointing out of the page (e.g., toward the viewer) and/or it is otherwise be difficult to view the scale of the axis due to the angle of the graph. In such instances, the coordinate values of this particular axis can be the same or similar to the coordinate values of the remaining two axis. For example, in graph 1210 of FIG. 9A, component 2 has coordinate values of between 600 and −600 (e.g., the same as components 0 and 1 and as shown by graph 1220). In graph 1220, component 1 has coordinate values of between 600 and −600 (as shown by graph 1210).

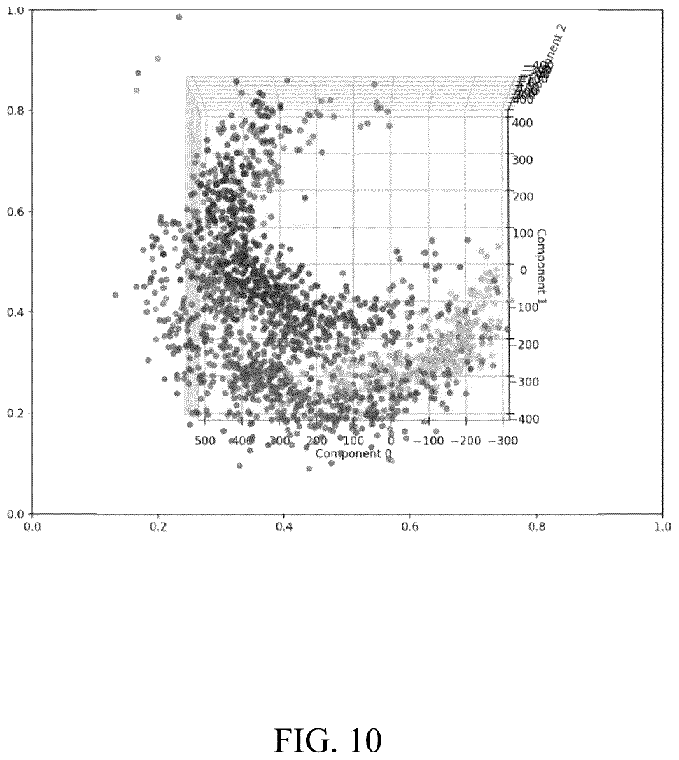

FIG. 10 illustrates example clusters of biosignal measures as projected in a trajectory along a torus, in accordance with the present disclosure. The same protocol was used to obtain the ECG biosignal measures as described in connection with FIGS. 8-9C. As shown by the graph of FIG. 10, the clusters appear at different phase angle and smaller amplitude as compared to FIG. 9A. In particular, the biostimulation signals applied at a pulse width of 500 μs (orange data points) had reduced variance relative to other clusters.

As shown by the experimental embodiments, taVNS had an effect on HRV, with a pulse width of 500 μs at 25 Hz appearing to be optimal for HRV. A 2π phase angle corresponded to about 190 samples or around 0.7 Hz, with the cleanest phase shift appearing with longer pulse width durations of 1 ms or 2 ms. The different waveforms having different pulse widths produced distinct effects on the torus, with a strong signal at about 0.8 Hz that is sensitive to taVNA. The size and angle of the clusters is dependent on stimulation properties and homeostatic context, with the variance of the clusters appearing to be correlated with HRV.

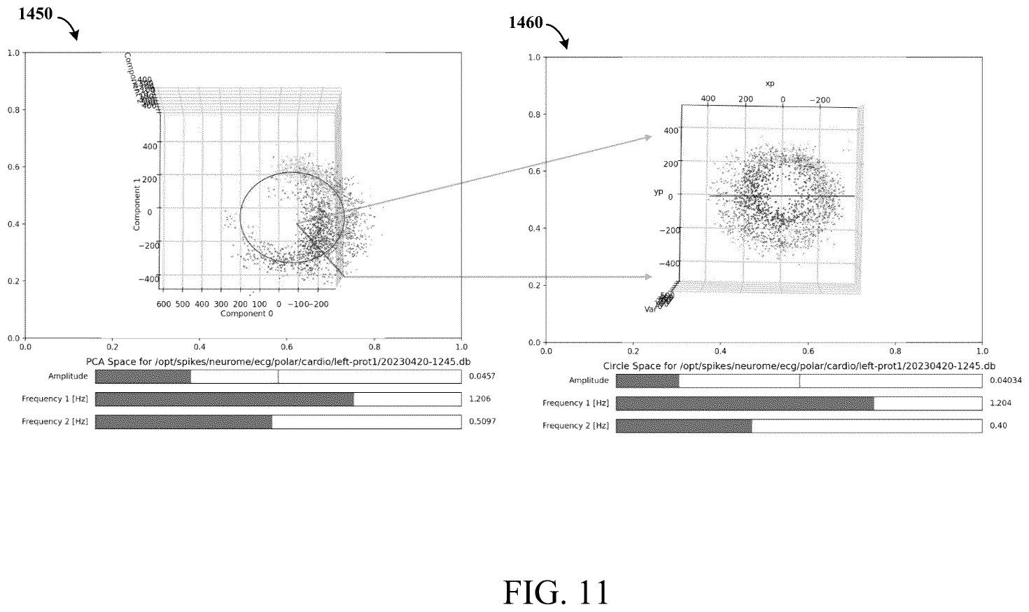

FIG. 11 illustrates an example of unwrapping the torus of biosignal measures, in accordance with the present disclosure. The graph 1450 includes a 3D PCA obtained from ECG measures responsive to the biostimulation signals applied, as described above. The graph 1460 includes a cross-section of the torus of the graph 1450, with points being plotted on the plane normal to the torus on the graph 1450 for all angles.

In the torus, the primary frequency (f1) represents the main cardiac cycle. A notch filter was used to see the clusters and dispersion of HRV. There is evidence of a secondary frequency (f2), as shown by the 2-torus embedded in 3D. The data shows that there can be a coupled quasi-Hamiltonian system with at least two oscillators when under perturbation by physical and neuronal forces. taVNA allowed for controlling perturbation of the biosystem, with different stimuli resulting in different trajectories on the torus. It is believed that the semicircular point clouds offered a strobed view of the torus.

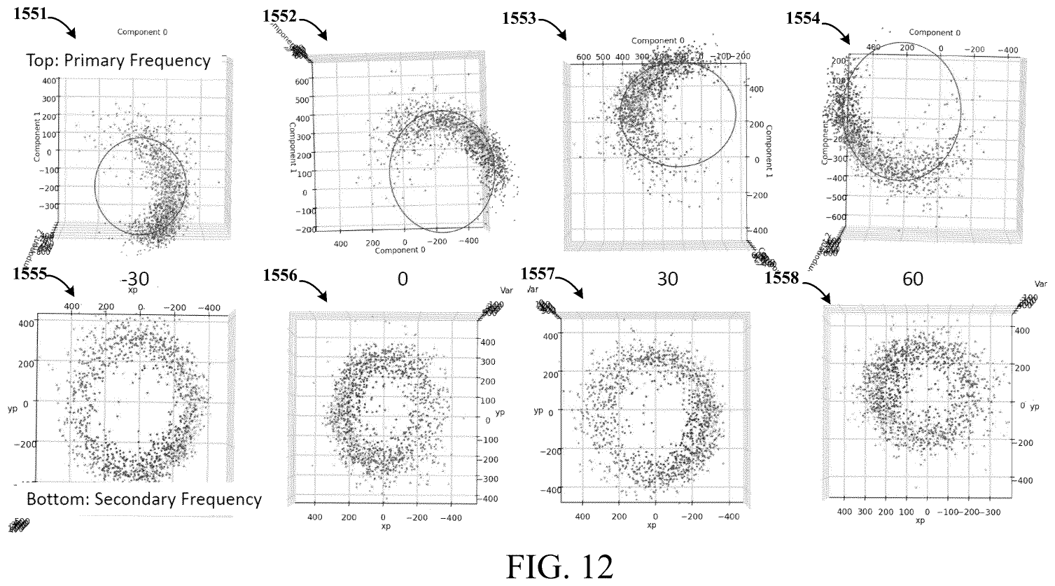

FIG. 12 illustrates an example of advancing the strobe of the biosignal measures, in accordance with the present disclosure. The same protocol was used to obtain the ECG biosignal measures as described in connection with FIGS. 8-9C, with the stimulus being applied to the right-side vagus nerve. The top graphs 1551, 1552, 1553, 1554 are 3D PCA plots with strobing the data by −30, 0, 30, and 60 on the first principal component (e.g., component 0). The bottom graphs 1555, 1556, 1557, 1558 are 3D PCA plots with strobing the data by −30, 0, 30, and 60 on the second principal component (e.g., component 1).

In various embodiments, the clusters were not apparent unless the data was strobed. The dataset windows were anchored to a central R peak, with each R peak getting its own data window. To strobe the data, the window timings were advanced or retarded with respect to the central R peak (e.g., the anchor), which slides the clusters in a clockwise or counter-clockwise direction. The default is zero, but with changing the timing, the clusters rotate along the primary frequency of the toroid. With the secondary frequency, the clusters flipped around. There appeared to be a higher frequency resonance, which is likely the finer PQ/ST structure of the cardiac cycle. The torus center wandered, in part to due to arbitrary initial conditions. It is believed that this is the projection of the main cardiac attractor onto a specific primary frequency, and at in this space, the effects on VNS were seen.

Some experimental embodiments were directed to applying taVNS to the left side, the right side, and/or bilaterally (e.g., left and right sides) and observing the differences.

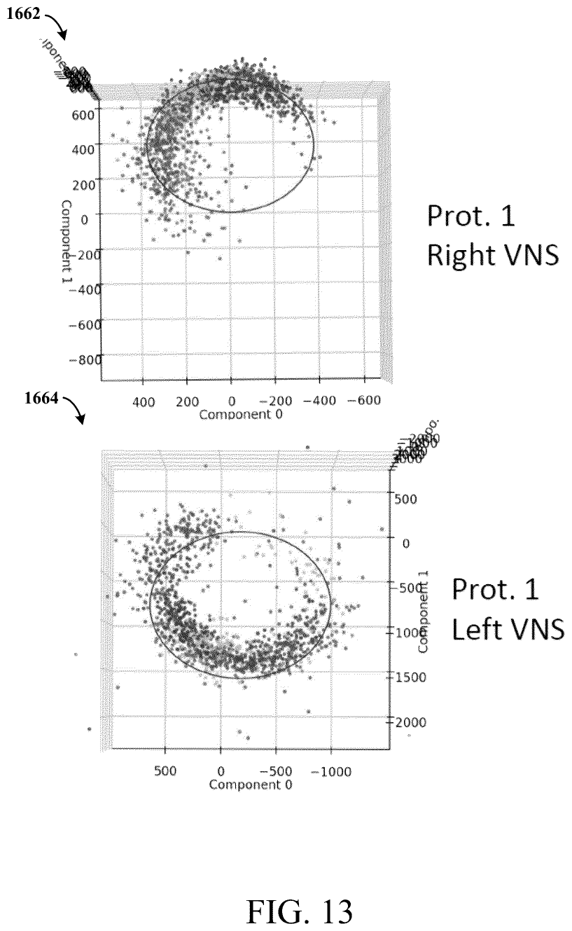

FIG. 13 illustrates an example of biosignal measures when stimulating on different sides of a subject, in accordance with the present disclosure. The same protocol was used to obtain the ECG biosignal measures as described in connection with FIGS. 8-9C. The top graph 1662 is a 3D PCA plot from biostimulation applied to the right side and the bottom graph 1664 is a 3D PCA plot from biostimulation applied to the left side. As shown, taVNA applied to the left side verses the right side reversed the cluster ordering on the torus. It is believed that taVNA applied to the left side decreased HR and taVNA applied to the right side increased HR.

In various embodiments, the biosystem of the subject can be sounded by applying biostimulation signals to a target having varying values for a stimulation parameter. The biostimulation signals included waveform files which can stored on a depository and that represent waveform delivery schedules and/or programs. The biostimulation signals were applied as a series of stimulation with washout periods between each epoch of stimulation, and with baseline biosignal measures collected for comparison. The baseline biosignal measures can be used to establish baseline observations without stimulation and used to assess the effect of stimulation. The biostimulation waveforms and schedules can affect the biosystem and produce different responses, which can be used to probe for the biosystem for homeostasis health. After collection, the biosignal measures are fit to a tori. With a cardiac biosystem and collection of ECG, the first principal component represented mean HR with taVNS perturbing HR. The second principal component represented PQST structures at around 1 Hz. Near-term variances of points on the tori provided a measure of HRV, which can be better than prior solutions that require long baselines and discard most of the data. The responsiveness of the cardiac biosystem can include changes to HR, variance, and the second principal component. The toroidal flow can reveal VNS parameter values that improve vagal tone and system responsiveness, such as compared to a baseline.

Although specific embodiments have been illustrated and described herein, a variety of alternate and/or equivalent implementations can be substituted for the specific embodiments shown and described without departing from the scope of the present disclosure. This application is intended to cover any adaptations or variations of the specific embodiments discussed herein.

1. A system comprising:
stimulation circuitry configured to output a plurality of biostimulation signals to a target of a subject;
sensor circuitry configured to obtain measures of a biosignal from the subject; and
processor circuitry configured to:
cause the stimulation circuitry to output the plurality of biostimulation signals to perturb a biosystem of the subject; and
quantify responsiveness of the biosystem to the perturbation based on the plurality of biostimulation signals and measures of the biosignal responsive to the plurality of biostimulation signals applied to the target.

2. The system of claim 1, further including memory circuitry in communication with the processor circuitry which stores a depository of the plurality of biostimulation signals, wherein each of the plurality of biostimulation signals represent a processed tissue signal as a sequence of at least one state corresponding to a set of state parameters and correlated with causing a particular physiological effect of perturbing the biosystem.

3. The system of claim 1, wherein the processor circuitry is configured to:
project the measures of the biosignal received from the sensor circuitry to provide a trajectory along a shape in phase space representing a biosignal cycle of the biosignal; and
based on clusters in the trajectory along the shape, quantify the responsiveness of the biosystem to the perturbation.

4. The system of claim 3, wherein the processor circuitry is configured to project the measures of the biosignal received from the sensor circuitry onto a Fourier basis to provide the trajectory along a torus in phase space representing the biosignal cycle of the biosignal.

5. The system of claim 3, wherein the processor circuitry is configured to output an indication of homeostatic context based on a size and angle of the clusters.

6. The system of claim 1, wherein the processor circuitry is configured to quantify the responsiveness of the biosystem for the subject over time and based on feedback, wherein the feedback is selected from at least one of:
additional measures of the biosignal, other physiological data, behavioral activity of the subject, environmental activity of the subject, and a combination thereof.

7. The system of claim 1, wherein the processor circuitry is configured to strobe the measures of the biosignal at a particular frequency.

8. The system of claim 1, wherein the plurality of biostimulation signals include different values for a stimulation parameter, wherein the stimulation parameter is selected from at least one of:
pulse frequency, duration, amplitude, duty cycle, pulse width, delivery portal, and a combination thereof.

9. The system of claim 1, wherein the processor circuitry includes a machine learning model which is trained using the plurality of biostimulation signals and measures of the biosignal to:
identify a transfer pattern of stimulation parameters that optimize an effect associated with the biosignal; and
predict the biosignal response and a homeostatic state, wherein the system outputs an indication of the biosignal response and the homeostatic state.

10. The system of claim 9, wherein the plurality of biostimulation signals include a plurality of neuromodulation signals applied to a nerve target of the subject.

11. The system of claim 1, wherein the processor circuitry is configured to establish a stimulus program that causes the stimulation circuitry to output an additional plurality of biostimulation signals to the subject or other subjects and timing for the additional plurality of biostimulation signals to achieve a goal associated with a homeostatic state.

12. The system of claim 1, wherein the processor circuitry includes a machine learning model which is trained to:
identify a first transfer pattern that maps the measures of a second biosignal and the plurality of biostimulation signals;
identify a second transfer pattern that maps the measures of the biosignal and the second biosignal; and
input the biosignal, as a proxy for the second biosignal, to the machine learning model and to predict an effect of an additional biostimulation signal on the second biosignal.

13. A method comprising:
applying a plurality of biostimulation signals to a target of a subject to perturb a biosystem of the subject;
receiving measures of a biosignal from the subject responsive to the plurality of biostimulation signals applied to the target; and
quantifying responsiveness of the biosystem to the perturbation based on the plurality of biostimulation signals and the measures of the biosignal.

14. The method of claim 13, wherein quantifying the responsiveness of the biosystem includes:
providing a trajectory of the measures of the biosignal along a shape in phase space representing a biosignal cycle of the biosignal; and
quantifying the responsiveness of the biosystem to the perturbation based on clusters in the trajectory along the shape.

15. The method of claim 14, wherein quantifying the responsiveness of the biosystem includes:
providing the trajectory along a torus in phase space representing the biosignal cycle of the biosignal by projecting the measures of the biosignal onto a Fourier basis; and
quantifying the responsiveness of the biosystem to the perturbation based on clusters in the trajectory along the torus.

16. The method of claim 13, further including identifying a transfer pattern of stimulation parameters that optimize an effect associated with the biosignal using a machine learning model which is trained using the plurality of biostimulation signals and the measures of the biosignal.

17. The method of claim 13, further including downloading the plurality of biostimulation signals from external memory circuitry, wherein each of the plurality of biostimulation signals represent a processed tissue signal as a sequence of at least one state corresponding to a set of state parameters and correlated with causing a particular physiological effect of perturbing the biosystem.

18. The method of claim 13, further including receiving baseline measures of the biosignal without biostimulation applied and comparing the baselines measures to the measures of the biosignal responsive to the plurality of biostimulation signals applied to the target to quantify the responsiveness of the biosystem.

19. The method of claim 13, further including:
receiving measures of a second biosignal from the subject;
using a machine learning model to:
identify a first transfer pattern that maps the measures of the second biosignal and the plurality of biostimulation signals; and
identify a second transfer pattern that maps the measures of the biosignal and the second biosignal; and
inputting the biosignal, as a proxy for the second biosignal, to the machine learning model and to predict an effect of an additional biostimulation signal on the second biosignal.

20. A non-transitory computer-readable storage medium comprising instructions that when executed cause processor circuitry to:
cause stimulation circuitry to output a plurality of biostimulation signals to a target of a subject to perturb a biosystem of the subject;
receive measures of a biosignal from the subject responsive to the plurality of biostimulation signals; and
quantify responsiveness of the biosystem to the perturbation based on the plurality of biostimulation signals and the measures of the biosignal responsive to the plurality of biostimulation signals.
